SEQUENCE LISTING (10 sequences)

SEQ ID NO: 1 - protein, 39 aa, Artificial Sequence
  Artificial mimic, synthesized.
  Exendin-4 amino acid (1)..(39)
HGEGTFTSDLSKQMEEEAVRLFIEWLKNGGPSSGAPPPS

SEQ ID NO: 2 - protein, 31 aa, Artificial Sequence
  Artificial mimic, synthesized.
  GLP-1 analogue amino acid (1)..(31): X or Xaa = Aib, alpha-aminoisobutyric acid, also known as 2-Aminoisobutyric acid.
HXEGTFTSDVSSYLEGQAAKEFIAWLVRGRG

SEQ ID NO: 3 - DNA, 24 bp, Artificial Sequence
  Artificial mimic, synthesized sense strand
  microRNA-34a (1)..(24): microRNA-34a, Sense strand.
uggcagugucuuagcugguugutt

SEQ ID NO: 4 - DNA, 24 bp, Artificial Sequence
  Artificial mimic, synthesized antisense.
  AntisensemicroRNA-34a (1)..(24): microRNA-34a antisense strand.
acaaccagcuaagacacugccatt

SEQ ID NO: 5 - protein, 32 aa, Artificial Sequence
  artificial synthesized peptide
  Human brain natriuretic peptide (1)..(32): Human brain natriuretic peptide
SPKMVQGSGCFGRKMDRISSSSGLGCKVLRRH

SEQ ID NO: 6 - protein, 8 aa, Artificial Sequence
  artificial synthesized peptide
  Octreotide (1)..(8): Octreotide with D-Phe and D-Trp amino acid residues D-Phe Cys Phe D-Trp Lys Thr Cys Thr.
FCFWKTCT

SEQ ID NO: 7 - DNA, 24 bp, Artificial Sequence
  artificial mimic, synthesized.
  Let-7 mimic (1)..(24): Let-7 mimic.
cuguacagccuccuagcuuucctt

SEQ ID NO: 8 - DNA, 25 bp, Artificial Sequence
  Artificial mimic, synthesized.
  miR-155 mimic (1)..(25): miR-155 mimic for to modulating immune responses.
uuaaugcuaauugugauaggggutt

SEQ ID NO: 9 - DNA, 21 bp, Artificial Sequence
  Synthessized human IRE1 siRNA
  human IRE1 siRNA (1)..(21): human IRE1 siRNA sense strand for immunomodulation and disease treatment.
  human IRE1 siRNA (1)..(21): human IRE1 siRNA sense strand for immunomodulation and disease treatment.
gcacgugaauugauagagatt

SEQ ID NO: 10 - protein, 28 aa, Homo sapiens
  human vasoactive intestinal polypeptide (VIP) (1)..(28): human vasoactive intestinal polypeptide (VIP)
HSDAVFTDNYTRLRKQMAVKKYLNSILN

(19) United States
(12) Patent Application Publication
(10) Pub. No.: US 2022/0339259 A1
(43) Pub. Date: Oct. 27, 2022

(54) ORALLY DELIVERED THERAPEUTICAL COMPOSITION AND USE THEREOF

(71) Applicant: Haifa SHEN, Woodcliff Lake, NJ (US)

(72) Inventor: Haifa SHEN, Woodcliff Lake, NJ (US)

(21) Appl. No.: 17/640,817

(22) PCT Filed: Sep. 6, 2020
(86) PCT No.: PCT/US20/49609

§ 371 Date: Mar. 5, 2022

Related U.S. Application Data
(60) Provisional application No. 62/896,662, filed on Sep. 6, 2019.

(51) Int. Cl. A61K 38/26; A61K 47/54; A61K 31/7088; A61K 38/22; A61K 47/36; A61K 9/127

(52) U.S. Cl. CPC A61K 38/26; A61K 47/542; A61K 31/7088; A61K 38/2278; A61K 47/36; A61K 47/548; A61K 9/1274

(57) ABSTRACT

This disclosure is directed to a pharmaceutical composition comprising a bioactive agent (active pharmaceutical ingredient, API) and at least one long chain fatty acid (LCFA), wherein the long chain fatty acid can comprise a carbon chain having at least 10 carbon atoms and can comprise a free carboxylic acid group or a salt thereof. The LCFA-conjugated active pharmaceutical ingredient (API) can be resistant to acid degradation in digestive system and facilitate the delivery of the API across the small intestinal epithelial cell membrane via fatty acid transport protein 4 (FATP4, also known as SLC27A4). The pharmaceutical composition can be formulated in acid-resistant (enteric-release) dosage forms for oral administration in patients. This disclosure is further directed to a process for producing the LCFA-conjugated bioactive agent including protein, polypeptide, small molecule drugs, DNA, RNA, oligonucleotide, or a combination thereof.

### FIELD OF THE DISCLOSURE

This disclosure is directed to a pharmaceutical composition suitable for oral administration for treating a disease in a subject in need thereof. This disclosure is further directed to a method for administering one or more therapeutic agents for treating a disease in the subject in need thereof.

### BACKGROUND

Oral delivery is the preferred route of drug administration; yet, it is not compatible with a large number of drug molecules such as the macromolecular biologics (therapeutic proteins, peptides, antibodies) and nucleotides (antisense oligonucleotides, small interfering ribonucleic acids, microRNAs, long and short non-coding RNAs, and mRNA therapeutics). For many small molecule drugs which are often packaged in orally deliverable dosage forms, there are needs to further improve bioavailability. The Bruton's tyrosine kinase (BTK) inhibitor ibrutinib is a perfect example. Although the small molecule compound has already been approved by the US Food and Drug Administration (FDA) for treatment of chronic lymphocytic leukemia and a number of other hematopoietic malignancies based on successful clinical trial results (Byrd, J. C., et al., N Engl J Med, 369(1):32-42, 2013; Burger, J. A., et al., N Engl J Med, 373(25):2425-2437, 2015; Dimopoulos, M. A., et al., N Engl J Med, 378(25):2399-2410, 2018), its oral bioavailability is only 3.9% in a fasting state and 8.4% in a fed state (de Vries, R., et al., Br J Clin Pharmacol, 81(2):235-45, 2016). One of the key factors that limit broad application of oral drug delivery is the existence of multiple physical, chemical and biological barriers in the gastrointestinal (GI) track including, but not limited to, the harsh acidic environment in the gastric fluid, multiple digestive enzymes, and a thick mucosal layer on top of epithelial cells with tight junctions. They either block drug transport or destroy drug molecules, resulting in diminished bioavailability of the therapeutic agents. Thus, it should be taken into consideration during drug design that a drug molecule or formulation is empowered with the capability to negotiate with the sequential barriers in order to achieve effective delivery efficiency, and hence optimal therapeutic efficacy (Blanco, E., Nat Biotechnol, 33(9):941-51, 2015).

Numerous attempts have been made to address the oral drug delivery barriers, yet most focused only on one barrier at a time and thus could not achieve the overall goal. For example, the blood glucose-lowering peptide drug semaglutide (a glucagon-like peptide 1 [GLP-1] analogue) has been co-formulated with an absorption enhancer, N-(8-[2-hydroxybenzoyl]amino) caprylate (SNAC). It is believed that SNAC facilitates semaglutide absorption in the stomach by triggering increase in pH in the gastric fluid and consequently enhancing solubility of the peptide drug and the protection against proteolytic enzymes (Granhall, C., et al., Clin Pharmacokinet, 58(6):781-791, 2019). However, oral bioavailability of this drug is still very low (1% to 2.5% in dogs and barely detectable in human) (Davies, M., et al., JAMA, 318(15):1460-1470, 2017).

Drug molecules have also been packaged in nanometer-size particles to protect degradation and to facilitate organ uptake. The best examples are therapeutic siRNA and microRNA (and siRNA mimics of microRNA). However, the bulky size of nanoparticles (usually in 50-200 nm range) is in itself a hindrance to drug transport since the nanoparticles can be easily blocked by the thick mucosal layer before getting contact with epithelial cells. In addition, after drug nanoparticles have been up taken by the epithelial cells through endocytosis or micropinocytosis, they enter the intracellular vesicular transport system where they need to escape endosomes quickly so that they are not destroyed by digestive enzymes and the acidic microenvironment. It has been revealed that only a very small percentage of nanoparticle-encapsulated therapeutic agents can reach the destiny organs/tissues (Wilhelm, S., et al., Nature Reviews Materials, 1(5), 2016; Park, K., Acs Nano, 7(9):7442-7447, 2013).

Two alternative approaches have been introduced to overcome the intestinal epithelial barrier. The first approach is to open the tight junctions between epithelial cells through co-administration of penetrate enhancers. However, this may cause physical disruption of the biological barriers and increase the risk of nonspecific absorption of toxins and pathogens from the GI track into the bloodstream (Moroz, E., Adv Drug Deliv Rev, 101:108-121, 2016; Menzel, C., et al., J Control Release, 277: 165-172, 2018; Buckley, S. T., et al., Sci Transl Med, 10(467), 2018). The second approach is to target receptors or transporters at the apical membrane of intestinal epithelial cells by loading drug molecules into ligand-conjugated nanoparticles (Pridgen, E. M., et al., Sci Transl Med, 5(213):213ra167, 2013). Some of the promising target receptors or transporters include the immunoglobulin Fc receptor (Pridgen, E. M., et al., Sci Transl Med, 5(213):213ra167, 2013), bile acid receptors (Schaap, F. G., Nat Rev Gastroenterol Hepatol, 11(1):55-67, 2014.) and the vitamin B12 receptor (Petrus, A. K., Angew Chem Int Ed Engl, 48(6):1022-8, 2009). However, Fc receptor interacts with the Fc portion of IgG in a pH-dependent manner, binding with high affinity in acidic environment which may cause harm to the therapeutic agent. On the other hand, bile acid transporters are distributed mainly in the ileum, but not the bulk of jejunum, so the therapeutic agent runs the risk of being degraded in the small intestine before reaching ileum. Thus, there is a strong demand to develop better and more effective strategies to enable oral delivery of therapeutic agents.

Triglycerides composed of long-chain fatty acids (LCFA) are a major part of Western diet (Bouchard-Mercier, A., et al., Nutrition Journal, 12, 2013). They are digested into fatty acids and glycerol in the GI track. The fatty acid transport protein 4 (FATP4, also known as SLC27A4) is the principal transporter in enterocytes that mediates LCFA absorption across the small intestine (Stahl, A., et al., Mol Cell, 4(3): 299-308, 1999). LCFA and glycerol are re-assembled into triglyceride and incorporated in the endoplasmic reticulum (ER) into chylomicrons which then exit the enterocyte and enter the lymphatic system. Efficiency of this route of transport is demonstrated by the over 95% LCFA absorption rate (Kalivianakis, M., et al., Am J Clin Nutr, 72(1):174-80, 2000). A prodrug involves a fatty acid carrier and a neuroactive drug dopamine was described in U.S. Pat. No. 4,939,174, issued on Jul. 3, 1990. The prodrug was, however, for intraperitoneal (IP) or subcutaneous (SC) injections and was speculated for oral administration. No data was available on the distribution or efficacy of oral use. Some drugs containing long chain fatty acid have been approved in the US. In 2019, Rybelsus, an oral version of the type 2 diabetes subcutaneous injection medication Semaglutide (trade names Ozempic) was approved in the United States as an oral drug. Rybelsus (Semaglutide) contains human glucagon-like peptide-1 (GLP-1) and a conjugated fatty acid with a long chain linker. Liraglutide is another long-acting, fatty acylated glucagon-like peptide-1 (GLP-1) analog administered via subcutaneous injection. Those drugs still suffer from inconvenience in administration and many other limitations.

Therefore, it is in need for a better composition and formulation for oral delivery of medications.

### SUMMARY

The present invention is directed to a pharmaceutical composition comprising a bioactive agent and at least one long chain fatty acid (LCFA), wherein the long chain fatty acid comprises a carbon chain having at least 10 carbon atoms comprising a first chain end and a second chain end, wherein the first chain end is covalently linked to the bioactive agent directly, or optionally, via a linker, and the second chain end comprises a free carboxylic acid group or a salt thereof.

In some cases, the pharmaceutical composition can comprise bioactive agent that comprises a polypeptide, a small molecule drug, a poly-nucleic acid, or a combination thereof, wherein said long chain fatty acid (LCFA) comprises C10 to C40 branched or linear saturated fatty acid, C10 to C40 branched or linear unsaturated fatty acid, or a combination thereof, and wherein said long chain fatty acid (LCFA) is linked to said bioactive agent via one or more functional groups that comprise C, N, O, P, S, an ether, an ester, an amide, a carbamate, a disulfide bond, a triazole, or a combination thereof, wherein the functional groups are on the bioactive agent, on the linker when present, or a combination thereof, and wherein the linker can comprise 0 to 40 atoms. In some cases, the bioactive agent can comprise a polypeptide and wherein the linker can comprise 0 to 10 atoms and the linker is free from glutamic acid, glutamic acid derivative, glutamine, glutamine derivative, or a combination thereof.

In some cases, the pharmaceutical composition can be formulated as an enteric-coated capsule, an enteric-coated tablet, an enteric-coated drug particle suspension, an enteric-coated drug powder, or a combination thereof.

The present invention is also directed to a process for producing a pharmaceutical composition comprising a bioactive agent covalently conjugated to at least one long chain fatty acid (LCFA), the process comprising:

a) providing a long chain dicarboxylic acid comprising a carbon chain having at least 10 carbon atoms, a first chain end having a first acid group and a second chain end having a second acid group, the first acid group and the second acid group are connected by the carbon chain;

b) reacting the first acid group, optionally via a linker, with the bioactive agent to produce the pharmaceutical composition;

wherein the second chain end in the pharmaceutical composition comprises a free carboxylic acid group or a salt thereof.

The present invention is further directed to a method for treating a disease of a subject in need thereof, the method comprises orally administering a pharmaceutically effective dosage of the pharmaceutical composition disclosed herein to the subject. The disease can be a metabolic disorder, a cancer, respiratory disease, depressing, diabetics, infectious disease, hypertensive, allergy, psychotic disorder, neurological disorder, arthritic disease, coagulation disease, ulcer, reflux, nausea, vitamins deficiency, heart failure, immunodeficiency, or a combination thereof.

### BRIEF DESCRIPTION OF THE FIGURES

FIG. 1. Schematic illustration of FATP4-mediated transport of LCFA-conjugated active pharmaceutical ingredient (API) across the small intestinal epithelial cell membrane. Once the LCFA-API conjugate gets in contact with FATP4-expressing cells (such as the epithelial cells in jejunum and ileum), the LCFA moiety interacts with the FATP4 transmembrane transporter and pulls the API tail into the cell membrane. The FATP4 facilitates LCFA the transmembrane process and transports LCFA-API into the cell. LCFA: long chain fatty acid. FATP4: fatty acid transport protein 4.

FIG. 2. Schematic illustration of an representative example of a structure of a pharmaceutical composition comprising LCFA-conjugated bioactive agent, such as active pharmaceutical ingredient (API). The active pharmaceutical ingredient (API) is covalently conjugated to LCFA through an optional linker. The API can be a therapeutic protein or peptide, a nucleic acid-based therapeutic agent, or a small molecule drug. The LCFA can be a branched or linear saturated fatty acid, mono-unsaturated fatty acid, or a poly-unsaturated fatty acid.

FIG. 3. Examples of FATP4 expression levels across organs in the GI track and in Caco-2 human colorectal cancer cells based on Western blot analysis. The tissue samples were collected from a mouse. FATP4 protein was detected with an anti-FATP4 antibody that recognized both human and murine FATP4 proteins, and β-actin served as a protein loading control for the analysis. The result indicates high levels of FATP4 expression in the jejunum and ileum, and a low level of FATP4 expression in duodenum. There is no detectable FATP4 expression in the stomach, cecum or colon. A high level of FATP4 expression was also detected in Caco-2 cells.

FIG. 4. Examples of immunostaining of FATP4 in murine duodenum, jejunum and colon tissue samples. FATP4 was stained in brown (indicated with arrows) with an anti-FATP4 antibody. Most epithelial cells in the duodenum were stained in light brown (indicated with arrows) indicating a moderate level of FATP4 expression. The epithelial cells in jejunum were stained dark brown (indicated with arrows), demonstrating a high level of FATP4 expression in these cells. Cells in the colon showed no stain, indicative of lack of FATP4 expression in colon tissue.

FIG. 5A-FIG. 5H. Examples of linkers suitable for linking LCFA and bioactive agent. A and B: an amide. C-D: an ester. E: a carbamate. F: a triazole. G: a disulfide bond. H: an ether. For convenience of chemical conjugation, a combination of linkers with a spacer in between can also be suitable.

FIG. 6A-FIG. 6D. Example of LCFA conjugated polypeptides. A: exendin-4 (Ex4, also known as exenatide, SEQ ID. 1) LCFA conjugate at both lysine residues (LYS¹² and LYS²⁷). B and C: Either one of the lysine residues LYS¹² or LYS²⁷ can be conjugated with a LCFA. D: GLP-1-Lys²⁶-LCFA conjugate. For GLP-1, the first histidine is traditionally counted as the 7th amino acid (His⁷), and the lysine residue as the 26th amino acid (Lys²⁶).

FIG. 7. An example of a representative high performance liquid chromatography (HPLC) analysis of a reaction mixture after chemical conjugation between a LCFA and Ex4. The retention time for an Ex4-LCFA conjugate at Lysine residue 27 (Ex4-Lys²⁷-LCFA) was 10.835 min, retention time for Ex4-Lys¹²-LCFA was 11.903 min, and retention time for Ex4-Lys¹²&²⁷-LCFA was 20.653. The respective peaks are pointed with arrows. Based on different retention times, these conjugates can be identified and purified with HPLC.

FIG. 8. An example of a representative HPLC profile of a purified Ex4-Lys²⁷-LCFA. Only one peak was shown in the region, indicating its high purity.

FIG. 9. An example of a representative mass spectrum (MS) profile of purified Ex4-Lys²⁷-LCFA. The molecular weight of the Ex4-Lys²⁷-LCFA conjugate was confirmed by the MS results.

FIG. 10. Examples of representative results of Caco-2 cell uptake. The fatty acid-conjugated Ex4 molecules were incubated with Alexa594 (fluorescent dye)-labeled Ex4-Lys²⁷-LCFA having 12, 16 and 18 carbon fatty acids (C12, C16, C18, respectively) and Ex4-Lys²⁷-SCFA (short chain fatty acid having 8 carbons, shown as C8) and were taken up by the cells. Levels of uptake were determined based on fluorescent intensity (MFI) inside the cells. The results indicate high uptake of Ex4-Lys²⁷-LCFA, but not Ex4-Lys²⁷-SCFA.

FIG. 11. Examples of representative glucose tolerance assay results. Glucose tolerance assay was done after oral gavage of free Ex4 and Ex4-Lys²⁷-LCFA in multiple groups of mice. Each mouse was treated orally with 100 μL 1M NaHCO₃ to neutralize acid in the gastric fluid. After 1 minute, the mice were treated with oral gavage (p.o.) of phosphate buffer saline (PBS), 100 μg free Ex4, or 100 μg Ex4-Lys²⁷-LCFA. The mice were then challenged with intraperitoneal injection of 2 g/kg glucose 1 hour after treatment. Mice treated with PBS or free Ex4 displayed high blood glucose levels. In comparison, mice treated with Ex4-Lys²⁷-LCFA showed a mild increase in blood glucose level 15 minutes after glucose challenge, and blood glucose level returned back to normal range quickly. The results indicate that LCFA-conjugated EX4, but not free Ex4, is effective in controlling blood glucose level after gavage in the acid-neutralized GI track.

FIG. 12. Additional examples of representative glucose tolerance assay results. Glucose tolerance assays were performed after treatment with free GLP-1 analogue or GLP-1-Lys²⁶-LCFA. Each mouse was treated orally with 100 μL 1M NaHCO₃ to neutralize acid in the gastric fluid as described before. After 1 minute, the mice were treated p.o. with PBS, 100 μg free GLP-1 analogue (GLP-1), or 100 μg GLP-1-Lys²⁶-LCFA. The mice were then challenged with intraperitoneal injection of 2 g/kg glucose 1 hour after treatment. Mice treated with PBS or free GLP-1 displayed high blood glucose levels. In comparison, mice treated with GLP-1-Lys26-LCFA showed a mild increase in blood glucose level 15 minutes after glucose challenge, and blood glucose level returned back to normal range afterwards. The results indicate LCFA-conjugated GLP-1 analogue, but not free GLP-1 analogue, is effective in controlling blood glucose level after gavage in the acid-neutralized GI track.

FIG. 13. Additional examples of representative glucose assay comparing different formulations. Glucose-lowering activities from GLP-1-Lys²⁶-LCFA after subcutaneous injection and oral gavage were compared. Mice were treated with PBS control, 3.3 μg unformulated GLP-1-Lys²⁶-LCFA by subcutaneous injection (s.c.), or 33 μg GLP-1-Lys²⁶-LCFA formulated in acid-resistant (enteric-release) hard gelatin capsules by oral gavage (p.o.). Both subcutaneously injected free GLP-1-Lys²⁶-LCFA and orally gavaged GLP-1-Lys²⁶-LCFA capsules showed comparable glucose-lowering activities. This result demonstrates that the enteric-coated GLP1-LCFA conjugate is effective in controlling blood glucose level.

FIG. 14. Examples of representative dosage effect on glucose-lowering activity from GLP-1-Lys²⁶-LCFA after oral gavage. GLP-1-Lys²⁶-LCFA molecules were pre-incubated with SiO₂ particles (which are one common excipient for drug tablets) and coated with Eudragit L-100 (which is a polymer for enteric coating). Mice were treated with PBS control, or increasing concentrations of GLP-1-Lys²⁶-LCFA by oral gavage. GLP-1-Lys²⁶-LCFA at 100 μg showed potent glucose-lowering activity. The results indicate that enteric coating is an effective method to protect LCFA-conjugated peptide from degradation by the acidic gastric fluid and digestive enzymes in the GI track.

FIG. 15A-FIG. 15C. Examples of representative structures and measurement data. A: chemical structures of ibrutinib. B: LCFA-conjugated ibrutinib (LCFA-ibrutinib). C: HPLC separation of free ibrutinib (shown as ibrutinib) from LCFA-ibrutinib.

FIG. 16A-FIG. 16B. Examples of representative HPLC data. Plasma ibrutinib levels were measured by HPLC 3 hours after mice were treated by oral gavage with an equal amount of free ibrutinib and LCFA-ibrutinib. The ibrutinib peaks are indicated with arrows. There was a 3-fold increase in plasma ibrutinib level in mice treated with LCFA-ibrutinib (B) compared with those treated with free ibrutinib (A) based on the measurements of the area under the curve (AUC).

FIG. 17A-FIG. 17B. Representative examples of structures of palmitate-conjugated microRNA-34a (miR-34a) mimic. A: The anti-sense strand of the miR-34a mimic duplex was chemically conjugated to LCFA and was annealed to the sense strand to form the double stranded RNA-LCFA complex. B: an examples of an antisense strand LCFA conjugate. The antisense strand is conjugated to palmitate through a linker and the sense strand is annealed to the antisense strand. The nucleotides in the sense and antisense strands are matched based on A-U and G-C pairing. There is a TT overhang at the 3′ end of both strands.

FIG. 18. Examples of representative measurement data of protein expression. Western blot was performed to measure the suppression of c-Myc expression by LCFA-conjugated miR-34a mimic 3 days after Caco-2 cells were treated. c-Myc is a known target of miR-34a. Its expression level was detected both after a short exposure (short expo) and a relatively long exposure (long expo). Suppression of c-Myc expression was detected only in cells treated with LCFA-miR-34a mimic (FA-miR-34a), but not the scramble microRNA mimic (Scr) or free miR-34a mimic control (miR-34a). The results indicate that LCFA-conjugated microRNA mimic, but not free microRNA mimic, can be taken up by the FATP4-expressing cells and can function properly inside the target cell.

### DETAILED DESCRIPTION

Following are more detailed descriptions of various concepts related to, and embodiments of, methods and apparatus according to the present disclosure. It should be appreciated that various aspects of the subject matter introduced above and discussed in greater detail below may be implemented in any of numerous ways, as the subject matter is not limited to any particular manner of implementation. Examples of specific implementations and applications are provided primarily for illustrative purposes.

As used herein,

The term “long-chain fatty acid”, “long chain fatty acid”, “LCFA”, “long-chain fatty acids”, “long chain fatty acids”, “LCFAs” or a grammatic variation thereof refers to branched or linear unsaturated fatty acid(s), branched or linear saturated fatty acid(s) having total 9 carbon or above (i.e., a total of 9 or above carbon atoms in the fatty acid molecule, such as C9-C40 fatty acids), a salt thereof, or a combination thereof. In some cases, a fatty acid(s) can comprise branched or linear unsaturated fatty acids, such as α-Linolenic acid (C18:3, Δ9,12,15, CH₃CH₂—CH═CHCH₂CH═CH—CH₂CH═CH(CH₂)₇—COOH), Stearidonic acid (C18:4, Δ6,9,12,15, CH₃CH₂CH═CH—CH₂CH═CH—CH₂CH═CHCH₂CH═CH—(CH₂)₄COOH), Eicosapentaenoic acid (C20:5, Δ5,8,11,14,17, CH₃CH₂CH═CH—CH₂CH═CH—CH₂CH═CHCH₂CH═CHCH₂CH═CH—(CH₂)₃COOH), Cervonic acid (C22:6, Δ4,7,10,13,16,19, CH₃CH₂CH═CH—CH₂CH═CHCH₂CH═CHCH₂CH═CH—CH₂—CH═CHCH₂CH═CH(CH₂)₂COOH), Linoleic acid (C18:2, Δ9,12, CH₃(CH₂)₄CH═CH—CH₂CH═CH(CH₂)₇COOH), Linolelaidic acid (C18:2, CH₃(CH₂)₄CH═CH—CH₂CH═CH(CH₂)₇COOH), γ-Linolenic acid (C18:3, Δ6,9,12, CH₃(CH₂)₄CH═CH—CH₂CH═CHCH₂CH═CH(CH₂)₄COOH), Dihomo-γ-linolenic acid (C20:3, Δ8,11,14, CH₃(CH₂)₄CH═CH—CH₂CH═CH—CH₂—CH═CH(CH₂)₆COOH), Arachidonic acid (C20:4, Δ5,8,11,14, CH₃(CH₂)₄CH═CH—CH₂CH═CH—CH₂CH═CH—CH₂CH═CH—(CH₂)₃COOH), Docosatetraenoic acid (C22:4, Δ7,10,13,16, CH₃(CH₂)₄—CH═CHCH₂CH═CHCH₂CH═CH—CH₂CH═CH(CH₂)₅—COOH), Palmitoleic acid (C16:1, Δ9, CH₃(CH₂)₅CH═CH(CH₂)₇COOH), Vaccenic acid (C18:1, Δ11, CH₃—(CH₂)₅CH═CH—(CH₂)₉COOH), Paullinic acid (C20:1, Δ13, CH₃(CH₂)₅CH═CH—(CH₂)₁₁COOH), Oleic acid (C18:1, Δ9, CH₃(CH₂)₇CH═CH—(CH₂)₇COOH), Elaidic acid (C18:1, Δ9, CH₃(CH₂)₇CH═CH(CH₂)₇COOH), Gondoic acid (C20:1, Δ11, CH₃(CH₂)₇CH═CH—(CH₂)₉COOH), Erucic acid (C22:1, Δ13, CH₃(CH₂)₇CH═CH—(CH₂)₁₁COOH), Nervonic acid (C24:1, Δ15, CH₃(CH₂)₇-CH═CH(CH₂)₁₃COOH), Mead acid (C20:3, Δ5,8,11, CH₃(CH₂)₇—CH═CH—CH₂CH═CH—CH₂CH═CH(CH₂)₃—COOH), branched or linear saturated fatty acid, such as, Undecanoic acid (C11:0, CH₃—(CH₂)₉COOH), Dodecanoic acid (C12:0, CH₃(CH₂)₁₀COOH), Tridecanoic acid (C13:0, CH₃(CH₂)₁₁COOH), Tetradecanoic acid (C14:0, CH₃(CH₂)₁₂COOH), Pentadecanoic acid (C15:0, CH₃(CH₂)₁₃COOH), Hexadecanoic acid (C16:0, CH₃(CH₂)₁₄COOH), Heptadecanoic acid (C17:0, CH₃ (CH₂)₁₅COOH), Octadec anoic acid (C18:0, CH₃(CH₂)₁₆COOH), Nonadecanoic acid (C19:0, CH₃(CH₂)₁₇COOH), Eicosanoic acid (C20:0, CH₃(CH₂)₁₈COOH), Heneicosanoic acid (C21:0, CH₃(CH₂)₁₉COOH), Docosanoic acid (C22:0, CH₃(CH₂)₂₀COOH), Tricosanoic acid (C23:0, CH₃(CH₂)₂₁COOH), Tetracosanoic acid (C24:0, CH₃(CH₂)₂₂COOH), Pentacosanoic acid (C25:0, CH₃(CH₂)₂₃COOH), Hexacosanoic acid (C26:0, CH₃(CH₂)₂₄COOH), Heptacosanoic acid (C27:0, CH₃(CH₂)₂₅COOH), Octacosanoic acid (C28:0, CH₃(CH₂)₂₆COOH), Nonacosanoic acid (C29:0, CH₃(CH₂)₂₇COOH), Triacontanoic acid (C30:0, CH₃(CH₂)₂₈COOH), Hentriacontanoic acid (C31:0, CH₃(CH₂)₂₉COOH), Dotriacontanoic acid (C32:0, CH₃(CH₂)₃₀COOH), Tritriacontanoic acid (C33:0, CH₃(CH₂)₃₁COOH), Tetratriacontanoic acid (C34:0, CH₃(CH₂)₃₂COOH), Pentatriacontanoic acid (C35:0, CH3(CH₂)₃₃COOH), Hexatriacontanoic acid (C36:0, CH₃(CH₂)₃₄COOH), Heptatriacontanoic acid (C37:0, CH₃(CH₂)₃₅COOH), Octatriacontanoic acid (C38:0, CH₃(CH₂)₃₆COOH), Nonatriacontanoic acid (C39:0, CH₃(CH₂)₃₇COOH), Tetracontanoic acid (C40:0, CH₃(CH₂)₃₈COOH), or a combination thereof. In some cases, branched fatty acids (also known as branched-chain fatty acid, BCFA) that found in animal and bacterial lipids can be suitable. In some cases, fatty carbon acyl chain can be saturated and can comprise methyl substituent groups. In some cases, unsaturated BCFAs found in marine animals can be suitable. In some cases, BCFA having branches other than methyl, such as those found in microbial lipids, can be suitable. In some cases, a branched fatty acid can comprise 2,3-Methylene suberic acid, 2,3-Methyleneglutaric acid, 2,4-Dimethyladipic acid, 2,4-Dimethylpimelic acid, 2-ethyl-2-butenoic acid, 2-Ethylglutaric acid, 2-Ethylhexanoic acid, 2-Methyl-4-pentenoic acid, 3,4-Methylene suberic acid, 3,4-Methyleneadipic acid, 3,4-Methyleneazelaic acid, 3,4-Methylenepimelic acid, 3,4-Methylenesebacic acid, 3-isopropylbut-3-enoic acid, 3-Methylazelaic acid, 3-Methylpimelic acid, 3-Methylsuberic acid, 4,8-dimethylnonanoic acid, other branched fatty acid known in the field, or a combination thereof.

As used herein, the term “Ex4-Lys¹²-LCFA” refers to a conjugated long-chain fatty acid and exendin-4 through the lysine residue 12 in exendin-4. Any of the LCFA disclosed herein can be suitable.

As used herein, the term “Ex4-Lys²⁷-LCFA” refers to a conjugated long-chain fatty acid and exendin-4 through the lysine residue 27 in exendin-4. Any of the LCFA disclosed herein can be suitable.

As used herein, the term “Ex4-Lys¹²&²⁷-LCFA” refers to a conjugated long-chain fatty acid and exendin-4 through the lysine residues 12 and 27 in exendin-4. Any of the LCFA disclosed herein can be suitable.

As used herein, the term “Ex4-Lys²⁷-SCFA” refers to a conjugated short-chain fatty acid (SCFA, such as C8 or less) and exendin-4 through the lysine residue 27 in exendin-4.

As used herein, the term “GLP-1-Lys²⁶-LCFA” refers to a conjugated long-chain fatty acid and GLP-1 analogue (with optional amino acid modifications from GLP-1) through the lysine residue 26 in the GLP-1 analogue. Any of the LCFA disclosed herein can be suitable.

As used herein, the amino acid sequence of Ex4 is: HIS GLY GLU GLY THR PHE THR SER ASP LEU SER LYS GLN MET GLU GLU GLU ALA VAL ARG LEU PHE ILE GLU TRP LEU LYS ASN GLY GLY PRO SER SER GLY ALA PRO PRO PRO SER (SEQ ID. 1). As used throughout this disclosure, each amino acid residue can be represented by a 3-letter symbol and can be in upper case letters, lower case letters or a combination thereof. Each amino acid can also be represented by a 1-letter symbol, either in upper case or lower case letter. A mixture of upper case letter or lower case letter can also be used.

As used herein, the amino acid sequence of GLP-1 analogue is: HIS^{T} AIB GLU GLY THR PHE THR SER ASP VAL SER SER TYR LEU GLU GLY GLN ALA ALA LYS²⁶ GLU PHE ILE ALA TRP LEU VAL ARG GLY ARG GLY (SEQ ID. 2). The first histidine has traditionally been counted as the 7th amino acid (His⁷), and the lysine residue as the 26th amino acid (Lys²⁶). AIB (also referred to as Aib) can also be represented by a standard amino acid modification code “Xaa” (SEQ ID. 2). AIB is alpha-aminoisobutyric acid as an amino acid substitution at position 8.

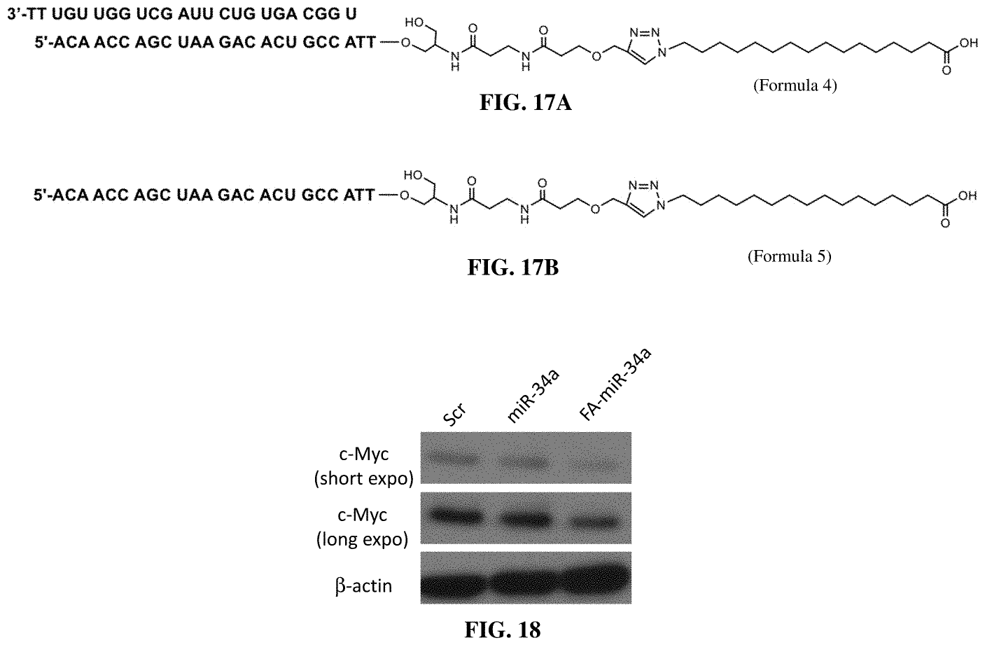

As used herein, the nucleic acid sequence of the sense strand of microRNA-34a (miR-34a) mimic is 5′-UGGCAGUGUCUUAGCUGGUUGUTT-3′ (SEQ ID. 3), and nucleotides 20 and 21 are methylated. The anti-sense strand of miR-34a mimic is 5′-ACAACCAGCUAAGACACUGCCATT-3′ (SEQ ID. 4). Nucleotides 1, 2, 3, 5, 7, 9, 11, 13, 15, 17 and 19 can be methylated, and the 3′ end is conjugated to palmitate through a linker or a linker with a spacer (FIG. 17A-FIG. 17B).

The term “pharmaceutically acceptable” in terms of an excipient, a carrier, a vehicle, an additive, or a filler (herein collectively referred to as “excipient”), is one that can be administered to a subject mammal or animal to provide an effective dose of the active ingredient and in compliance with government regulations. Any pharmaceutical composition disclosed herein can further comprise an excipient, wherein the excipient can comprise those listed in the US FDA inactive ingredient database (IID) or reagents determined to be generally regarded as safe (GRAS). An excipient can be a pharmaceutically acceptable material that can be administered to a subject to provide an effective dose of an active ingredient and in compliance with government regulations. An excipient can be selected from a detergent, a buffer, a phosphate, a salt, a water, a solvent, a filler, an inorganic compound, an organic compound, a synthetic polymer, a biopolymer, a sugar, a starch, a polysaccharide, SiO₂, Eudragit L-100-coated SiO₂, or a combination thereof.

In some cases, this disclosure is directed to a pharmaceutical composition comprising a bioactive agent and at least one long chain fatty acid (LCFA), wherein the long chain fatty acid comprises a carbon chain having at least 10 carbon atoms comprising a first chain end and a second chain end, wherein the first chain end is covalently linked to the bioactive agent directly, or optionally, via a linker, and the second chain end comprises a free carboxylic acid group or a salt thereof.

The bioactive agent can comprise a polypeptide, a small molecule drug, a poly-nucleic acid, or a combination thereof, wherein the long chain fatty acid (LCFA) can comprise a C10 to C40 branched or linear saturated fatty acid, C10 to C40 branched or linear unsaturated fatty acid, or a combination thereof, and wherein the long chain fatty acid (LCFA) is linked to the bioactive agent via one or more functional groups that comprise C, N, O, P, S, an ether, an ester, an amide, a carbamate, a disulfide bond, a triazole, or a combination thereof, wherein the functional groups are on the bioactive agent, on the linker when present, or a combination thereof, and wherein the linker can comprise 0 to 40 atoms. The LCFA can comprise a linear saturated fatty acid in one example, a branched saturated fatty acid in another example, a linear unsaturated fatty acid in yet another example, a branched unsaturated fatty acid in yet another example, and a combination thereof in yet another example. In some cases, at least one of the LCFA comprises a C10 to C40 fatty acid, a C11 to C40 fatty acid, a C12 to C40 fatty acid, a C13 to C40 fatty acid, a C14 to C40 fatty acid, a C15 to C40 fatty acid, a C16 to C40 fatty acid, a C17 to C40 fatty acid, a C18 to C40 fatty acid, a C19 to C40 fatty acid, a C20 to C40 fatty acid, a C21 to C40 fatty acid, a C22 to C40 fatty acid, a C23 to C40 fatty acid, a C24 to C40 fatty acid, a C25 to C40 fatty acid, a C26 to C40 fatty acid, a C27 to C40 fatty acid, a C28 to C40 fatty acid, a C29 to C40 fatty acid, a C30 to C40 fatty acid, a C31 to C40 fatty acid, a C32 to C40 fatty acid, a C33 to C40 fatty acid, a C34 to C40 fatty acid, a C35 to C40 fatty acid, a C36 to C40 fatty acid, a C37 to C40 fatty acid, a C38 to C40 fatty acid, a C39 to C40 fatty acid, or a combination thereof. In some cases, at least one of the LCFA comprises a C10 to C40 fatty acid, a C10 to C39 fatty acid, a C10 to C38 fatty acid, a C10 to C37 fatty acid, a C10 to C36 fatty acid, a C10 to C35 fatty acid, a C10 to C34 fatty acid, a C10 to C33 fatty acid, a C10 to C32 fatty acid, a C10 to C31 fatty acid, a C10 to C30 fatty acid, a C10 to C29 fatty acid, a C10 to C28 fatty acid, a C10 to C27 fatty acid, a C10 to C26 fatty acid, a C10 to C25 fatty acid, a C10 to C24 fatty acid, a C10 to C23 fatty acid, a C10 to C22 fatty acid, a C10 to C21 fatty acid, a C10 to C20 fatty acid, a C10 to C19 fatty acid, a C10 to C18 fatty acid, a C10 to C17 fatty acid, a C10 to C16 fatty acid, a C10 to C15 fatty acid, a C10 to C14 fatty acid, a C10 to C13 fatty acid, a C10 to C12 fatty acid, a C10 to C11 fatty acid, or a combination thereof. In some cases, at least one of the LCFA comprises a C16 to C28 fatty acid, a C17 to C28 fatty acid, a C18 to C28 fatty acid, a C19 to C28 fatty acid, a C20 to C28 fatty acid, a C21 to C28 fatty acid, a C22 to C28 fatty acid, a C23 to C28 fatty acid, a C24 to C28 fatty acid, a C25 to C28 fatty acid, a C26 to C28 fatty acid, or a C27 to C28 fatty acid. In some cases, at least one of the LCFA comprises a C16 to C24 fatty acid, a C17 to C24 fatty acid, a C18 to C24 fatty acid, a C19 to C24 fatty acid, or a C20 to C24 fatty acid.

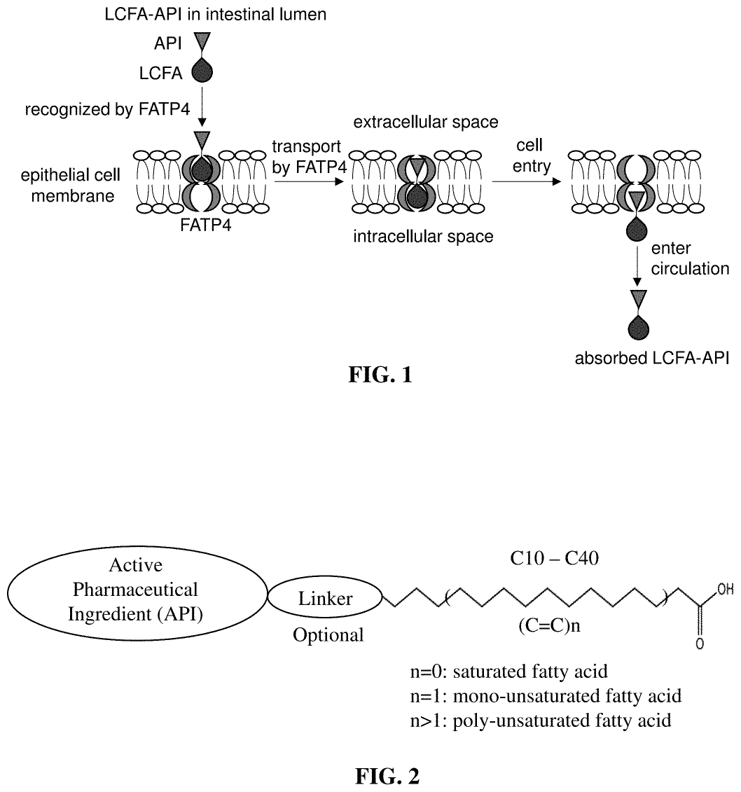

Not wishing to be bound by a particular theory or a mechanism, Applicant believes the covalently linked bioactive agent and LCFA conjugate, herein also referred to as active pharmaceutical ingredient (API)-LCFA conjugate or LCFA-API, can be recognized by fatty acid transport protein 4 (FATP4) in FATP4-expressing cells, such as the epithelial cells in duodenum, jejunum and ileum and transported by FATP4 across the cell membrane to enter the cells (FIG. 1). A schematic structure of an example of an LCFA-API is illustrated in FIG. 2. The long chain fatty acid can be a linear or branched saturate or an unsaturated C10 to C40 fatty acid, or a combination thereof. The free carboxylic acid group or a salt thereof can be recognized by the FATP4 and can be a required for FATP4 mediated cross cell membrane transport. The pharmaceutical composition disclosed herein that comprises the free carboxylic acid group or a salt thereof can provide an advantage for delivering the bioactive agent to cells, tissues or organs that comprise FATP4 expression, such as small intestine, Duodenum and Jejunum, not in the tissues or organs lacking the FATP4 express, such as stomach.

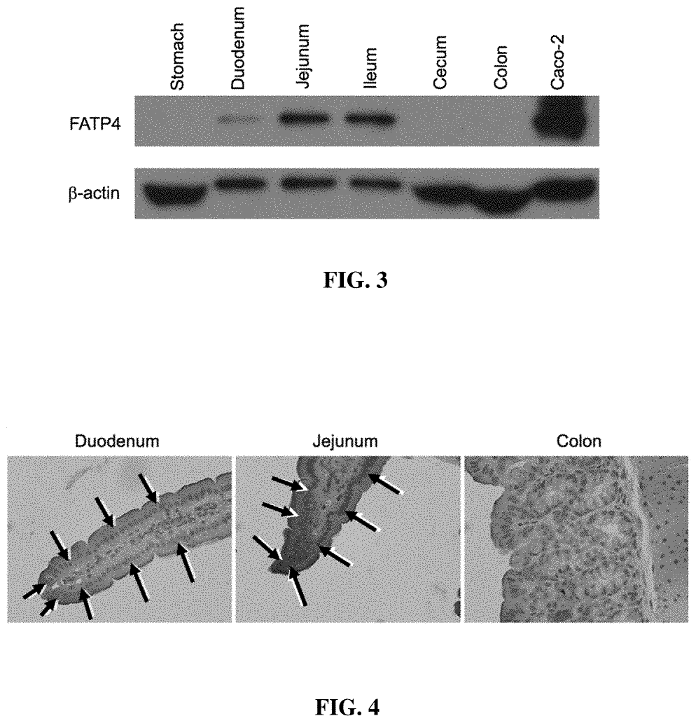

FATP4 protein level in various tissues and organs can be measured using Western blot or other suitable methods as determined appropriate by those in the field. Some representative protein expression levels of FATP4 are shown in FIG. 3. Immunostaining can also be used to determine the location of the FATP4 protein in cells, tissues or organs. Some representative immunostaining images are shown in FIG. 4 showing FATP4 presence in the Duodenum and Jejunum, but not in the Colon (FIG. 4).

In some cases, the pharmaceutical composition disclosed herein can be targeted for delivering the bioactive agent to cells, tissues or organs that comprise FATP4 expression. In some cases, the pharmaceutical composition disclosed herein can be targeted for delivering the bioactive agent to small intestine, Duodenum or Jejunum in a subject, and wherein cells, tissues or organs lacking the FATP4 express in the subject can be free from the bioactive agent. In some cases, the cells, tissues or organs lacking the FATP4 expression in the subject can comprise stomach or stomach cells.

The polypeptide can comprise amino acid, modified amino acid, amino acid analogue, or a combination thereof, and the long chain fatty acid (LCFA) can be linked to the polypeptide at one or more —N—, —NH—, —NH₂, —S—, —SH, —OH, —COO— or a combination thereof, of one or more residues of the amino acid, modified amino acid, amino acid analogue, or a combination thereof, of the polypeptide. In some cases, the LCFA can be linked to the —NH₂ group of a lysine residue of the polypeptide. In some cases, the LCFA can be linked to the —SH group of a cystine residue of the polypeptide. In some cases, the LCFA can be linked to the —NH₂ group at the N-terminal amino acid residue. In some cases, the LCFA can be linked to the —COOH group at the C-terminal amino acid residue.

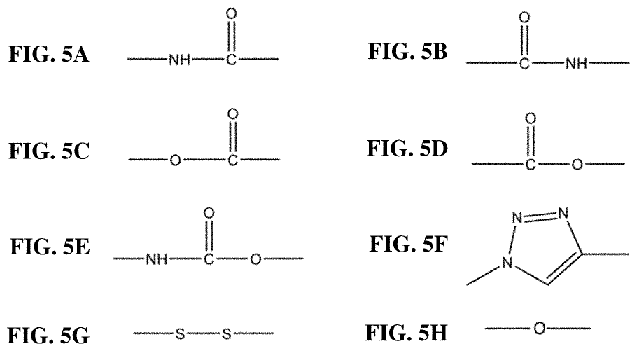

In some cases, the pharmaceutical composition can comprise a linker that can comprise 0 to 40 atom and can comprise an amide (FIG. 5A and FIG. 5B), an ester (FIG. 5C and FIG. 5D), a carbamate (FIG. 5E), a triazole (FIG. 5F), a disulfide bond (FIG. 5G), an ether (FIG. 5H), or a combination thereof. The pharmaceutical composition can further comprise one or more carbon chains, spacers, or a combination thereof, in combination with the aforementioned linker. In some cases, the pharmaceutical composition can be free from a linker, wherein the LCFA and the bioactive agent are directly linked via groups within the respective molecules, i.e., the link comprises 0 atoms. In some cases, the linker can comprise 1 to 40 atoms, 2 to 40 atoms, 3 to 40 atoms, 4 to 40 atoms, 5 to 40 atoms, 6 to 40 atoms, 7 to 40 atoms, 8 to 40 atoms, 9 to 40 atoms, 10 to 40 atoms, 11 to 40 atoms, 12 to 40 atoms, 13 to 40 atoms, 14 to 40 atoms, 15 to 40 atoms, 16 to 40 atoms, 17 to 40 atoms, 18 to 40 atoms, 19 to 40 atoms, 20 to 40 atoms, 21 to 40 atoms, 22 to 40 atoms, 23 to 40 atoms, 24 to 40 atoms, 25 to 40 atoms, 26 to 40 atoms, 27 to 40 atoms, 28 to 40 atoms, 29 to 40 atoms, 30 to 40 atoms, 31 to 40 atoms, 32 to 40 atoms, 33 to 40 atoms, 34 to 40 atoms, 35 to 40 atoms, 36 to 40 atoms, 37 to 40 atoms, 38 to 40 atoms or 39 to 40 atoms. In some cases, the linker can comprise 1 to 39 atoms, 1 to 38 atoms, 1 to 37 atoms, 1 to 36 atoms, 1 to 35 atoms, 1 to 34 atoms, 1 to 33 atoms, 1 to 32 atoms, 1 to 31 atoms, 1 to 30 atoms, 1 to 29 atoms, 1 to 28 atoms, 1 to 27 atoms, 1 to 26 atoms, 1 to 25 atoms, 1 to 24 atoms, 1 to 23 atoms, 1 to 22 atoms, 1 to 21 atoms, 1 to 20 atoms, 1 to 19 atoms, 1 to 18 atoms, 1 to 17 atoms, 1 to 16 atoms, 1 to 15 atoms, 1 to 14 atoms, 1 to 13 atoms, 1 to 12 atoms, 1 to 11 atoms, 1 to 10 atoms, 1 to 9 atoms, 1 to 8 atoms, 1 to 7 atoms, 1 to 6 atoms, 1 to 5 atoms, 1 to 4 atoms, 1 to 3 atoms, or 1 to 2 atoms.

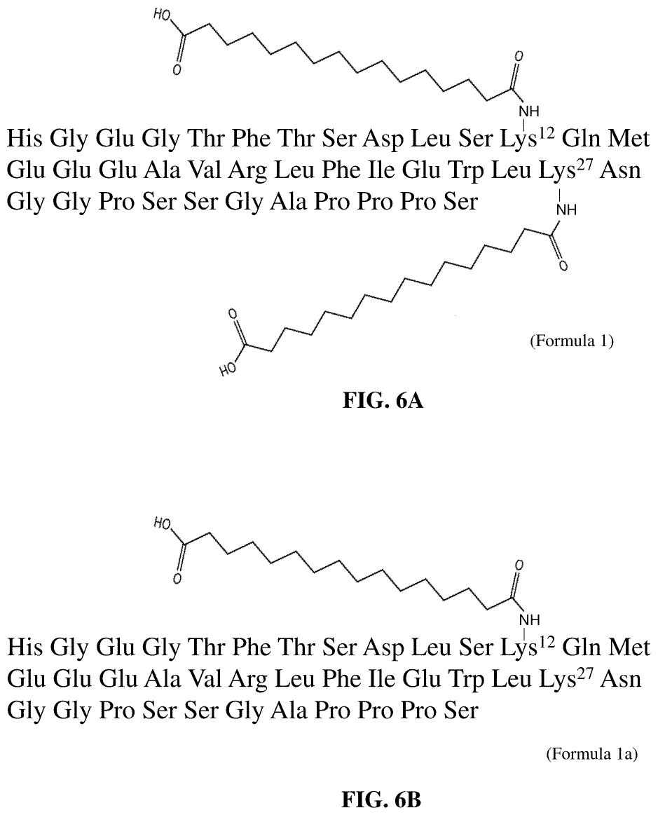

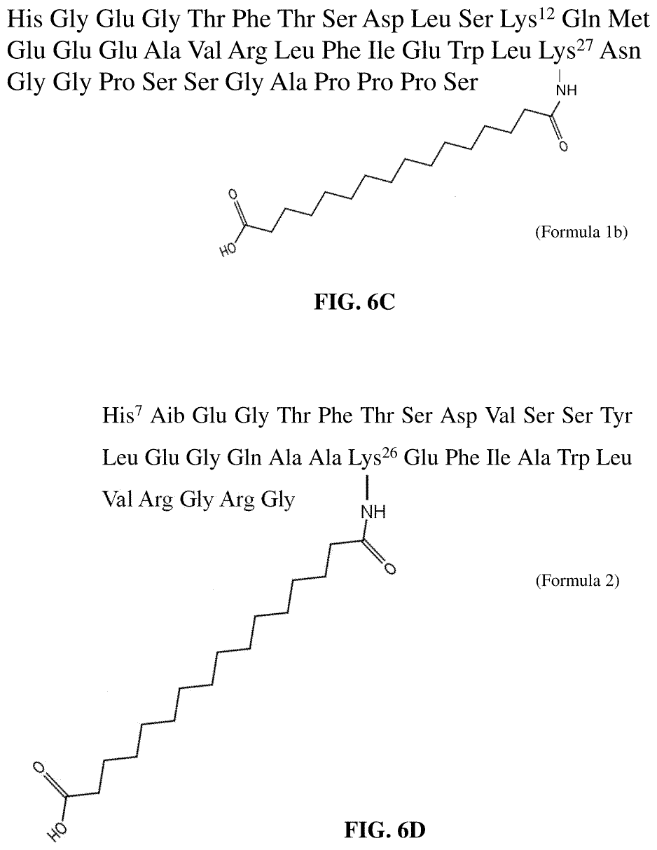

In some cases, the bioactive agent can comprise a polypeptide and wherein the linker can comprise 0 to 10 atoms and the linker can be free from glutamic acid, glutamic acid derivative, glutamine, glutamine derivative, or a combination thereof. In some cases, the bioactive agent can comprise a polypeptide that comprises 15 to 100 amino acid residues, wherein the polypeptide comprises at least one lysine residue, and wherein the LCFA is directly linked to the at least lysine residue of the polypeptide free from a linker (FIG. 6A-FIG. 6D). In some cases, the pharmaceutical composition can be produced by reacting a C10 to C40 long chain dicarboxylic acid with one or more lysine residues of a polypeptide that comprises one or more lysine residues producing the pharmaceutical composition comprising the polypeptide covalently linked to a long chain fatty acid directly and free from a linker.

Not wishing to be bound by a particular theory or mechanism, Applicant believe that a pharmaceutical composition comprises a shorter linker that comprises 0 to 10 atoms or free from a linker can provide an advantage for easy manufacturing, lower manufacturing cost and potentially lower side effects or complications.

In some cases, suitable to any of the pharmaceutical compositions of this disclosure, the bioactive agent can comprise a glucagon or a derivative thereof, a glucagon analogue or a derivative thereof, a glucagon-like peptide 1 (GLP-1) or a derivative thereof, exendin-4 or a derivative thereof, insulin or a derivative thereof, human brain natriuretic peptide or a derivative thereof, octreotide or a derivative thereof, human vasoactive intestinal peptide (VIP) or a derivative thereof, or a combination thereof. As used herein, a bioactive agent derivative thereof can comprise a part of the bioactive agent, a fragment of the bioactive agent, one or more modified amino acid residues, one or more substituted amino acid residues, a peptide having at least 75% homology to the bioactive agent, a peptide having substantially the same function as the bioactive agent, or a combination thereof. The bioactive agent can be linked to the LCFA via the linker that comprises 0 to 10 atoms and the linker can be free from glutamic acid, glutamic acid derivative, glutamine, glutamine derivative, or a combination thereof, if present. The bioactive agent can be linked to the LCFA without a linker.

In some cases, the pharmaceutical composition can comprise the bioactive agent and the long chain fatty acid (LCFA) having the

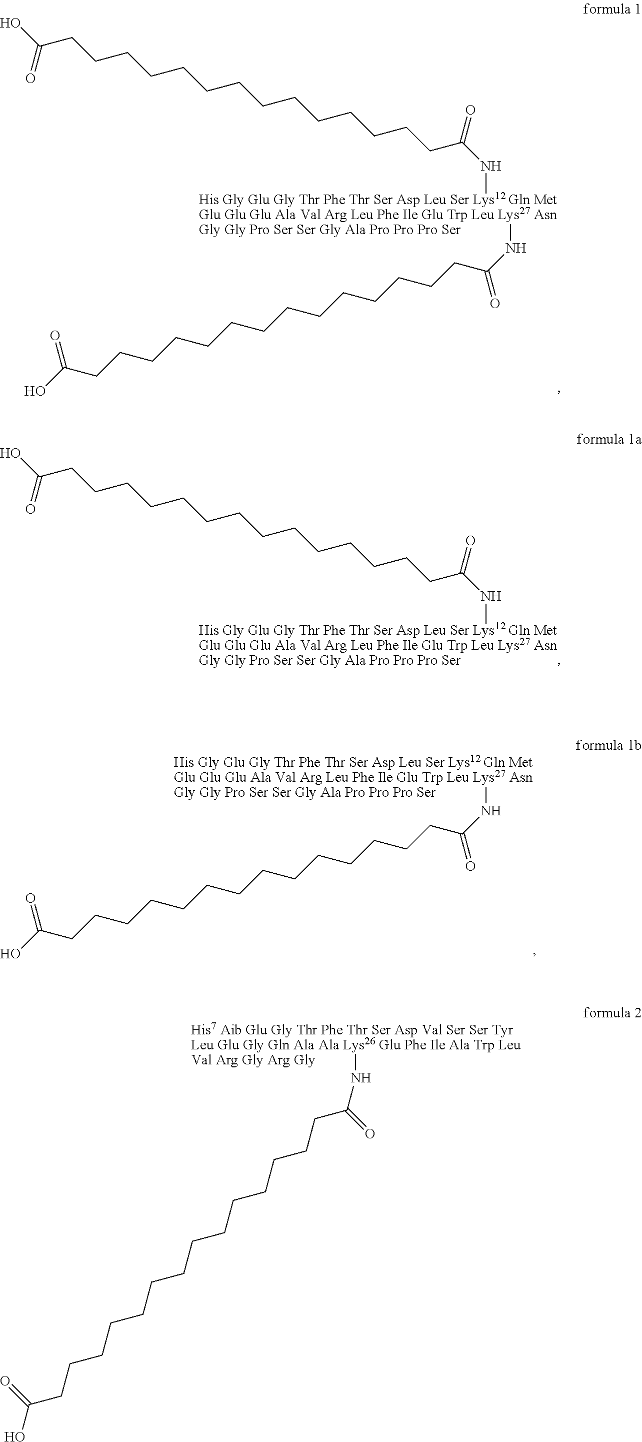

or a combination thereof. Representative examples are shown in FIG. 6A-FIG. 6C (Ex4-LCFA) and FIG. 6D (GLP-1-LCFA).

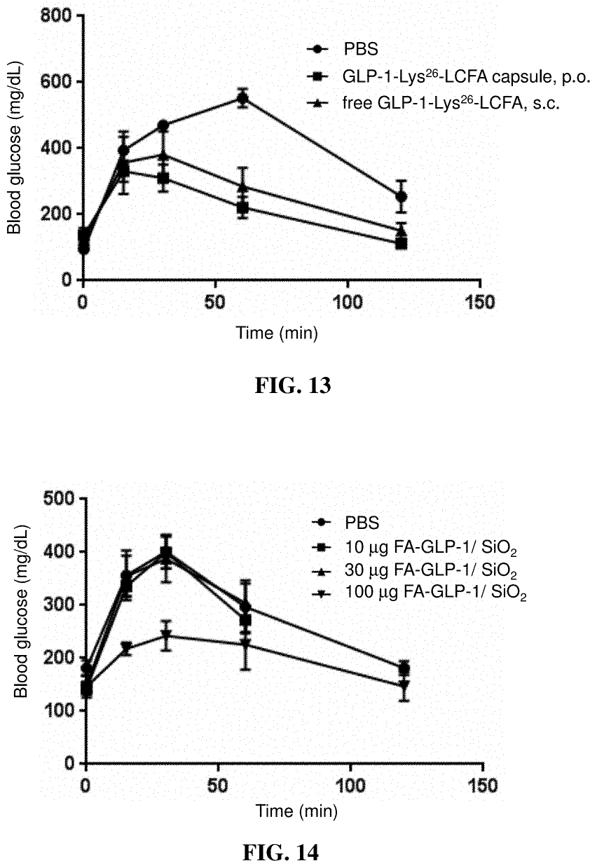

In some cases, the pharmaceutical compositions of this disclosure can comprise a glucagon-like peptide 1 (GLP-1) linked to a LCFA at Lys²⁶ (GLP-1-Lys²⁶-LCFA). The GLP-1 can be linked to the LCFA via a linker that can comprise 0 to 10 atoms and the linker can be free from glutamic acid, glutamic acid derivative, glutamine, glutamine derivative, or a combination thereof. In some cases, the pharmaceutical composition can comprise a GLP-1 linked directly to the LCFA via an amino group of Lys²⁶ without a linker, wherein the long chain fatty acid (LCFA) can comprise a C10 to C40 branched or linear saturated fatty acid, C10 to C40 branched or linear unsaturated fatty acid, or a combination thereof. Data shown in FIG. 13 and FIG. 14 demonstrate that enteric-coated GLP1-LCFA conjugates disclosed herein can be effective in controlling blood glucose level. A GLP-1 derivative thereof can comprise a part of the GLP-1 peptide, a fragment of the GLP-1 peptide, one or more modified amino acid residues, one or more substituted amino acid residues, a peptide having at least 75% homology to the GLP-1 peptide, a peptide having substantially the same function as the GLP-1 peptide, or a combination thereof.

In some cases, the pharmaceutical compositions of this disclosure can comprise an exendin-4 (Ex4) linked to a LCFA at Lys¹² (Ex4-Lys¹²-LCFA), an exendin-4 (Ex4) linked to a LCFA at Lys²⁷ (Ex4-Lys²⁷-LCFA), an exendin-4 (Ex4) linked to a LCFA at both Lys¹² and Lys²⁷ (Ex4-Lys¹²/Lys²⁷-LCFA), or a combination thereof. The Ex4 can be linked to the LCFA via the linker that can comprise 0 to 10 atoms and the linker can be free from glutamic acid, glutamic acid derivative, glutamine, glutamine derivative, or a combination thereof. In some cases, the pharmaceutical composition can comprise Ex4 directly to the LCFA via an amino group of Lys¹², an amino group of Lys²⁷, both amino groups of Lys¹² and Lys²⁷, without a linker, wherein the long chain fatty acid (LCFA) can comprise a C10 to C40 branched or linear saturated fatty acid, a C10 to C40 branched or linear unsaturated fatty acid, or a combination thereof. An Ex4 derivative thereof can comprise a part of the Ex4 peptide, a fragment of the Ex4 peptide, one or more modified amino acid residues, one or more substituted amino acid residues, a peptide having at least 75% homology to the Ex4 peptide, a peptide having substantially the same function as the Ex4 peptide, or a combination thereof.

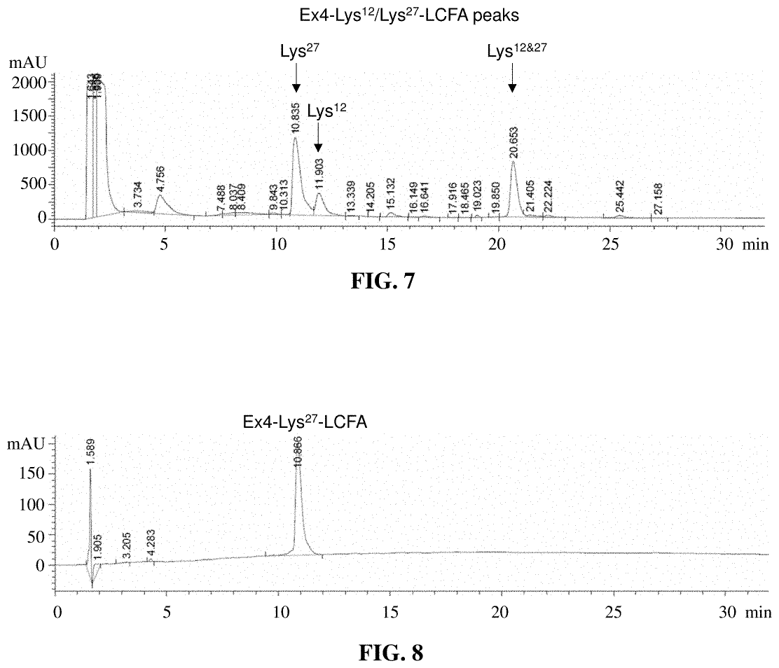

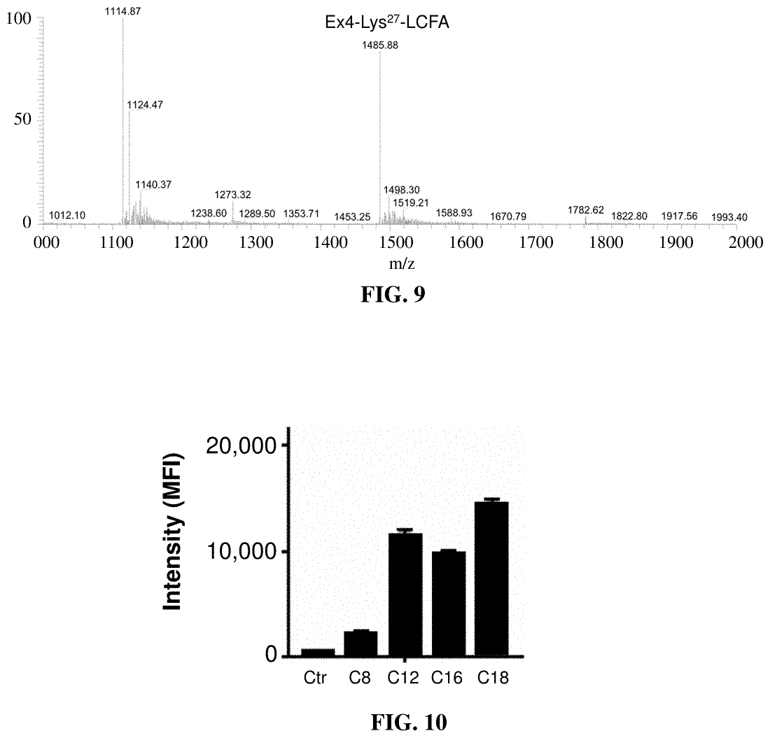

In some cases, the bioactive agent can comprise exendin-4 (Ex4) (SEQ ID. 1). An example of representative Ex4-LCFA is shown as Ex4-Lys¹²&²⁷-LCFA in FIG. 6A. Some examples of measurement data on Ex4 (free Ex4), Ex4-Lys¹²&²⁷-LCFA, Ex4-Lys²⁷-LCFA, Ex4-Lys¹²-LCFA are shown in FIG. 7, FIG. 8, FIG. 9, FIG. 10 and FIG. 11.

In some cases, the pharmaceutical composition comprises purified Ex4-Lys²⁷-LCFA, wherein the LCFA can be a C10 to C22 linear saturated fatty acid, a C10 to C22 linear unsaturated fatty acid, a C10 to C23 linear saturated fatty acid, a C10 to C23 linear unsaturated fatty acid, a C10 to C24 linear saturated fatty acid, a C10 to C24 linear unsaturated fatty acid, a C10 to C25 linear saturated fatty acid, a C10 to C25 linear unsaturated fatty acid, a C10 to C26 linear saturated fatty acid, a C10 to C26 linear unsaturated fatty acid, a C10 to C27 linear saturated fatty acid, a C10 to C27 linear unsaturated fatty acid, a C10 to C28 linear saturated fatty acid, a C10 to C28 linear unsaturated fatty acid, a C10 to C29 linear saturated fatty acid, a C10 to C29 linear unsaturated fatty acid, a C10 to C30 linear saturated fatty acid, a C10 to C30 linear unsaturated fatty acid, a C10 to C31 linear saturated fatty acid, a C10 to C31 linear unsaturated fatty acid, a C10 to C32 linear saturated fatty acid, a C10 to C32 linear unsaturated fatty acid, a C10 to C33 linear saturated fatty acid, a C10 to C33 linear unsaturated fatty acid, a C10 to C34 linear saturated fatty acid, a C10 to C34 linear unsaturated fatty acid, a C10 to C35 linear saturated fatty acid, a C10 to C35 linear unsaturated fatty acid, a C10 to C36 linear saturated fatty acid, a C10 to C36 linear unsaturated fatty acid, a C10 to C37 linear saturated fatty acid, a C10 to C37 linear unsaturated fatty acid, a C10 to C38 linear saturated fatty acid, a C10 to C38 linear unsaturated fatty acid, a C10 to C39 linear saturated fatty acid, a C10 to C39 linear unsaturated fatty acid, or a C10 to C40 linear saturated fatty acid, or a C10 to C40 linear unsaturated fatty acid.

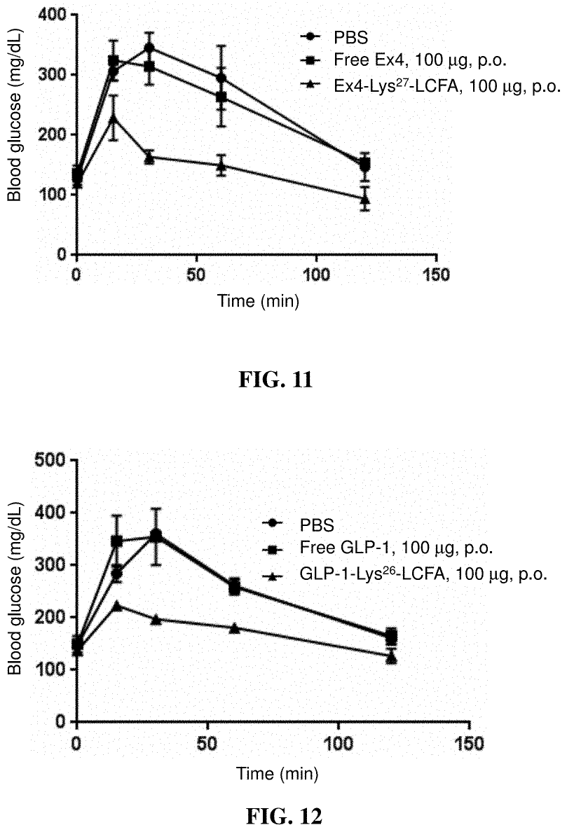

In some cases, the bioactive agent can comprise glucagon-like peptide 1 (GLP-1) or a GLP-1 derivative with modified amino acid Aib (SEQ ID. 2). An example of representative GLP-1-LCFA is shown as GLP-1-Lys²⁶-LCFA (FIG. 6B). The LCFA can be linked to the lysine residue as the 26th amino acid position. Some examples of measurement data on GLP-1 (free GLP-1), GLP-1-Lys²⁶-LCFA, GLP-1-Lys²⁶-LCFA capsule, and dosage effects are shown in FIG. 12, FIG. 13 and FIG. 14.

In some cases, the bioactive agent can comprise human brain natriuretic peptide for heart failure treatment. The human brain natriuretic peptide can have an amino acid sequence: Ser Pro Lys Met Val Gln Gly Ser Gly Cys Phe Gly Arg Lys Met Asp Arg Ile Ser Ser Ser Ser Gly Leu Gly Cys Lys Val Leu Arg Arg His (SEQ ID. 5). There can be a disulfide bond between the two Cys residues. A human brain natriuretic peptide derivative thereof can comprise a part of the human brain natriuretic peptide, a fragment of the human brain natriuretic peptide, one or more modified amino acid residues, one or more substituted amino acid residues, a peptide having at least 75% homology to the human brain natriuretic peptide, a peptide having substantially the same function as the human brain natriuretic peptide, or a combination thereof.

In some cases, the bioactive agent can comprise octreotide for cancer treatment. The octreotide can have an amino acid sequence: D-Phe Cys Phe D-Trp Lys Thr Cys Thr (SEQ ID. 6). There can be D-Phe and D-Trp amino acid residues in the peptide. There can be a disulfide bond between the two Cys residues. An octreotide derivative thereof can comprise a part of the octreotide, a fragment of the octreotide, one or more modified amino acid residues, one or more substituted amino acid residues, a peptide having at least 75% homology to the octreotide, a peptide having substantially the same function as the octreotide, or a combination thereof.

In some cases, the bioactive agent can comprise human vasoactive intestinal peptide (VIP) for restoring organ function. A representative human VIP can have a peptide sequence: His Ser Asp Ala Val Phe Thr Asp Asn Tyr Thr Arg Leu Arg Lys Gln Met Ala Val Lys Lys Tyr Leu Asn Ser Ile Leu Asn (SEQ ID. 10). Chemical conjugation of the VIP and LCFA can be linked at one or more of the Lys residues, the N-terminal —NH₂ group, the C-termina —COOH group, or a combination thereof. A human vasoactive intestinal peptide derivative thereof can comprise a part of the human vasoactive intestinal peptide, a fragment of the human vasoactive intestinal peptide, one or more modified amino acid residues, one or more substituted amino acid residues, a peptide having at least 75% homology to the human vasoactive intestinal peptide, a peptide having substantially the same function as the human vasoactive intestinal peptide, or a combination thereof.

In some cases, the bioactive agent can comprise a metabolic drug, a cancer drug, a chemotherapeutic drug, analgesics/antipyretics, anesthetics, antiasthmatics, antidepressants, antidiabetics, antifungal agents antihypertensive agents, antipsychotic agents, antimanic agents, antiarrhythmics, antiarthritic agents, antigout agents, anticoagulants, thrombolytic agents, antifibrinolytic agents, hemorheologic agents, antiplatelet agents, anticonvulsants, antiparkinson agents, antihistamines/antipruritics, agents useful for calcium regulation, antibacterial agents, antiviral agents, antimicrobials, antibiotics, anti-infectives, corticosteroids, thyroid hormones, hypoglycemic agents selected from insulin, recombinant insulin, glyburide, chlorpropamide, glipizide, tolbutamide, tolazamide, or a combination thereof, hypolipidemic agents selected from clofibrate, dextrothyroxine sodium, probucol, lovastatin, niacin or a combination thereof, antiulcer or antireflux agents, antinauseants or antiemetics, vitamins, mitotane, visadine, halonitrosoureas, anthrocyclines, ellipticine, or a combination thereof. If a bioactive agent comprises one or more of the functional groups that comprise C, N, O, P, S, an ether, an ester, an amide, a carbamate, a disulfide bond, a triazole, or a combination thereof, the LCFA can be linked to one or more of the functional groups of the bioactive agent. If the bioactive agent is free from one or more of the functional groups mentioned above, a linker, a spacer, or a combination thereof, can be added using the chemistry known to those in the field and the LCFA can be linked to the linker.

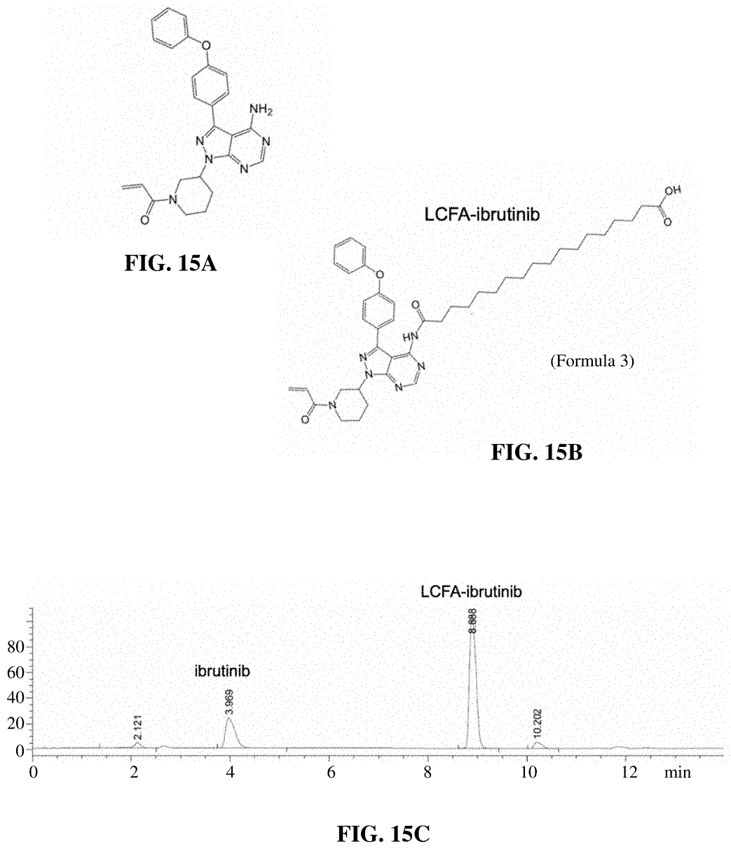

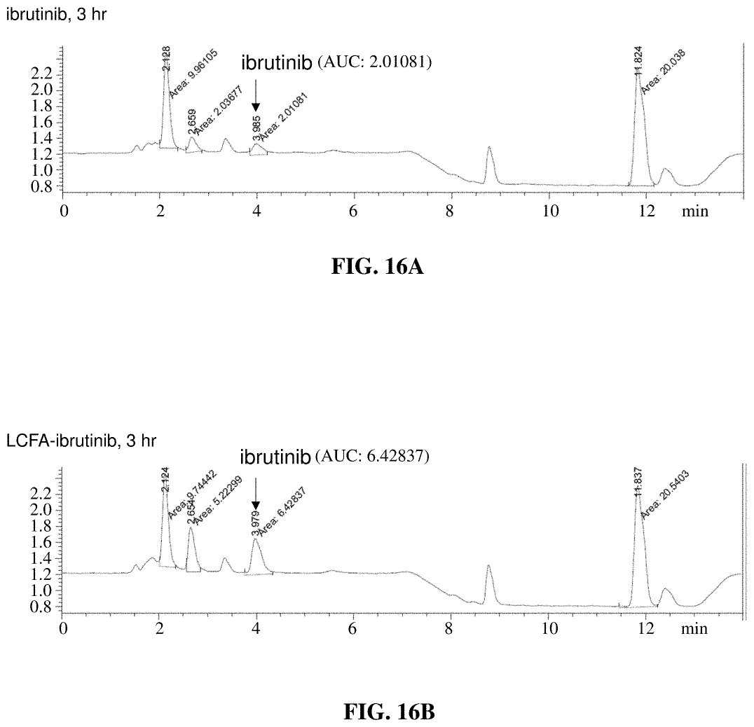

In some cases, the bioactive agent can comprise an ibrutinib. Examples of chemical structures of ibrutinib, LCFA-ibrutinib and measurement data are shown in FIG. 15A, FIG. 15B, FIG. 15C, FIG. 16A and FIG. 16B, respectively.

In some cases, the pharmaceutical composition can comprise the bioactive agent and the long chain fatty acid (LCFA) having the formula 3:

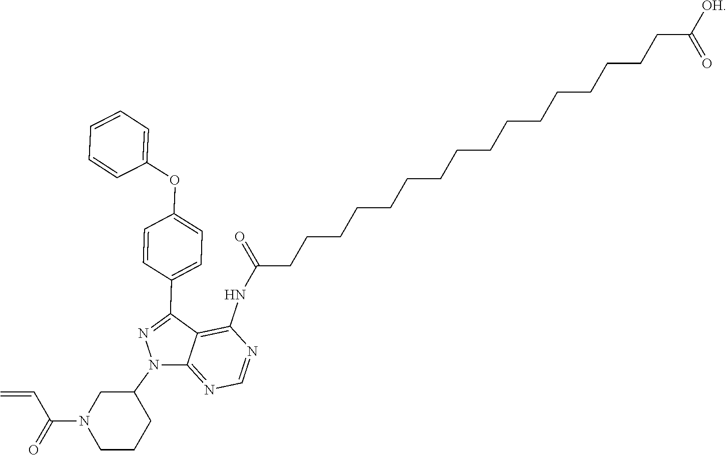 [SMILES: C=CC(=O)N1CCCC(n2nc(-c3ccc(Oc4ccccc4)cc3)c3c(NC(=O)CCCCCCCCCCCCCCCCC(=O)O)ncnc32)C1]

In some cases, the bioactive agent can comprise, small molecule drug, such as, a chemotherapeutic drug, analgesics/antipyretics; anesthetics such as cyclopropane, enflurane, halothane, isoflurane, methoxyflurane, nitrous oxide, propofol; antiasthmatics such as azelastine, ketotifen, traxanox, amlexanox, cromolyn, ibudilast, montelukast, nedocromil, oxatomide, pranlukast, seratrodast, suplatast tosylate, tiaramide, zafirlukast, zileuton, beclomethasone, budesonide, dexamethasone, flunisolide, triamcinolone acetonide; antibiotics such as neomycin, streptomycin, chloramphenicol, cephalosporin, ampicillin, penicillin, tetracycline; antidepressants such as nefopam, oxypertine, doxepin hydrochloride, amoxapine, trazodone hydrochloride, amitriptyline hydrochloride, maprotiline hydrochloride, phenelzine sulfate, desipramine hydrochloride, nortriptyline hydrochloride, tranylcypromine sulfate, fluoxetine hydrochloride, doxepin hydrochloride, imipramine hydrochloride, imipramine pamoate, nortriptyline, amitriptyline hydrochloride, isocarboxazid, trimipramine maleate, protriptyline hydrochloride; antidiabetics such as biguanides, hormones, sulfonylurea derivatives; antifungal agents such as griseofulvin, ketoconazole, amphotericin B, nystatin, candicidin; antihypertensive agents such as propanolol, propafenone, oxyprenolol, nifedipine, reserpine, trimethaphan camsylate, phenoxybenzamine hydrochloride, pargyline hydrochloride, deserpidine, diazoxide, guanethidine monosulfate, minoxidil, rescinnamine, sodium nitroprusside, rauwolfia serpentina, alseroxylon, phentolamine mesylate, reserpine; anti-inflammatories such as non-steroidal compounds, such as, indomethacin, naproxen, ibuprofen, ramifenazone, piroxicam, and steroidal compounds, such as, cortisone, dexamethasone, fluazacort, hydrocortisone, prednisolone, prednisone; antineoplastics such as adriamycin, cyclophosphamide, actinomycin, bleomycin, daunorubicin, doxorubicin, epirubicin, mitomycin, methotrexate, fluorouracil, carboplatin, carmustine (BCNU), methyl-CCNU (semustine), cisplatin, etoposide, interferons, camptothecin and derivatives thereof, phenesterine, Taxol and derivatives thereof, taxotere and derivatives thereof, vinblastine, vincristine, tamoxifen, etoposide, piposulfan; antianxiety agents such as lorazepam, buspirone hydrochloride, prazepam, chlordiazepoxide hydrochloride, oxazepam, clorazepate dipotassium, diazepam, hydroxyzine pamoate, hydroxyzine hydrochloride, alprazolam, droperidol, halazepam, chlormezanone, dantrolene; immunosuppressive agents such as cyclosporine, azathioprine, mizoribine, FK506 (tacrolimus); antimigraine agents such as ergotamine tartrate, propanolol hydrochloride, isometheptene mucate, dichloralphenazone; sedatives/hypnotics such as barbiturates, pentobarbital, pentobarbital sodium, secobarbital sodium, benzodiazapines, flurazepam hydrochloride, triazolam, tomazeparm, midazolam hydrochloride; antianginal agents such as β-adrenergic blockers, calcium channel blockers, nifedipine, diltiazem hydrochloride and nitrates, nitroglycerin, isosorbide dinitrate, pentaerythritol tetranitrate, erythrityl tetranitrate; antipsychotic agents such as haloperidol, loxapine succinate, loxapine hydrochloride, thioridazine, thioridazine hydrochloride, thiothixene, fluphenazine hydrochloride, fluphenazine decanoate, fluphenazine enanthate, trifluoperazine hydrochloride, chlorpromazine hydrochloride, perphenazine, lithium citrate, prochlorperazine; antimanic agents such as lithium carbonate; antiarrhythmics such as bretylium tosylate, esmolol hydrochloride, verapamil hydrochloride, amiodarone, encamide hydrochloride, digoxin, digitoxin, mexiletine hydrochloride, disopyramide phosphate, procainamide hydrochloride, quinidine sulfate, quinidine gluconate, quinidine polygalacturonate, flecamide acetate, tocamide hydrochloride, lidocaine hydrochloride; antiarthritic agents such as phenylbutazone, sulindac, penicillamine, salsalate, piroxicam, azathioprine, indomethacin, meclofenamate sodium, gold sodium thiomalate, ketoprofen, auranofin, aurothioglucose, tolmetin sodium; antigout agents such as colchicine, allopurinol; anticoagulants such as heparin, heparin sodium, warfarin sodium; thrombolytic agents such as urokinase, streptokinase, altoplase; antifibrinolytic agents such as aminocaproic acid; hemorheologic agents such as pentoxifylline; antiplatelet agents such as aspirin, empirin, ascriptin; anticonvulsants such as valproic acid, divalproate sodium, phenyloin, phenyloin sodium, clonazepam, primidone, phenobarbitol, phenobarbitol sodium, carbamazepine, amobarbital sodium, methsuximide, metharbital, mephobarbital, mephenyloin, phensuximide, paramethadione, ethotoin, phenacemide, secobarbitol sodium, clorazepate dipotassium, trimethadione; antiparkinson agents such as ethosuximide; antihistamines/antipruritics such as hydroxyzine hydrochloride, diphenhydramine hydrochloride, chlorpheniramine maleate, brompheniramine maleate, cyproheptadine hydrochloride, terfenadine, clemastine fumarate, triprolidine hydrochloride, carbinoxamine maleate, diphenylpyraline hydrochloride, phenindamine tartrate, azatadine maleate, tripelennamine hydrochloride, dexchlorpheniramine maleate, methdilazine hydrochloride, trimprazine tartrate; agents useful for calcium regulation such as calcitonin, parathyroid hormone; antibacterial agents such as amikacin sulfate, aztreonam, chloramphenicol, chloramphenicol palmitate, chloramphenicol sodium succinate, ciprofloxacin hydrochloride, clindamycin hydrochloride, clindamycin palmitate, clindamycin phosphate, metronidazole, metronidazole hydrochloride, gentamicin sulfate, lincomycin hydrochloride, tobramycin sulfate, vancomycin hydrochloride, polymyxin B sulfate, colistimethate sodium, colistin sulfate; antiviral agents such as interferon γ, zidovudine, amantadine hydrochloride, ribavirin, acyclovir; antimicrobials such as cephalosporins, cefazolin sodium, cephradine, cefaclor, cephapirin sodium, ceftizoxime sodium, cefoperazone sodium, cefotetan disodium, cefutoxime azotil, cefotaxime sodium, cefadroxil monohydrate, ceftazidime, cephalexin, cephalothin sodium, cephalexin hydrochloride monohydrate, cefamandole nafate, cefoxitin sodium, cefonicid sodium, ceforanide, ceftriaxone sodium, ceftazidime, cefadroxil, cephradine, cefuroxime sodium, penicillins, ampicillin, amoxicillin, penicillin G benzathine, cyclacillin, ampicillin sodium, penicillin G K, penicillin V K, piperacillin sodium, oxacillin sodium, bacampicillin hydrochloride, cloxacillin sodium, ticarcillin disodium, azlocillin sodium, carbenicillin indanyl sodium, penicillin G procaine, methicillin sodium, nafcillin sodium, erythromycins, erythromycin ethylsuccinate, erythromycin, erythromycin estolate, erythromycin lactobionate, erythromycin stearate, erythromycin ethylsuccinate, tetracyclines, tetracycline hydrochloride, doxycycline hyclate, minocycline hydrochloride; anti-infectives such as GM-CSF; bronchodilators such as sympathomimetics, epinephrine hydrochloride, metaproterenol sulfate, terbutaline sulfate, isoetharine, isoetharine mesylate, isoetharine hydrochloride, albuterol sulfate, albuterol, bitolterol, mesylate isoproterenol hydrochloride, terbutaline sulfate, epinephrine bitartrate, metaproterenol sulfate, epinephrine, epinephrine bitartrate; anticholinergic agents such as ipratropium bromide; xanthines such as aminophylline, dyphylline, metaproterenol sulfate, aminophylline; mast cell stabilizers such as cromolyn sodium; inhalant corticosteroids such as flunisolide, beclomethasone dipropionate monohydrate, salbutamol, beclomethasone dipropionate (BDP), ipratropium bromide, budesonide, ketotifen, salmeterol, xinafoate, terbutaline sulfate, triamcinolone, theophylline, nedocromil sodium, metaproterenol sulfate, albuterol, flunisolide; hormones such as androgens, danazol, testosterone cypionate, fluoxymesterone, ethyltostosterone, testosterone enanthate, methyltestosterone, fluoxymesterone, testosterone cypionate, estrogens, estradiol, estropipate, conjugated estrogens, progestins, methoxyprogesterone acetate, norethindrone acetate, corticosteroids, triamcinolone, betamethasone, betamethasone sodium phosphate, dexamethasone, dexamethasone sodium phosphate, dexamethasone acetate, prednisone, methylprednisolone acetate suspension, triamcinolone acetonide, methylprednisolone, prednisolone sodium phosphate methylprednisolone sodium succinate, hydrocortisone sodium succinate, methylprednisolone sodium succinate, triamcinolone hexacatonide, hydrocortisone, hydrocortisone cypionate, prednisolone, fluorocortisone acetate, paramethasone acetate, prednisolone tebulate, prednisolone acetate, prednisolone sodium phosphate, hydrocortisone sodium succinate, thyroid hormones, levothyroxine sodium; hypoglycemic agents such as human insulin, purified beef insulin, purified pork insulin, glyburide, chlorpropamide, glipizide, tolbutamide, tolazamide; hypolipidemic agents such as clofibrate, dextrothyroxine sodium, probucol, lovastatin, niacin; proteins such as DNase, alginase, superoxide dismutase, lipase; nucleic acids such as sense or anti-sense nucleic acids encoding any therapeutically useful protein, including any of the proteins or polypeptides described herein; agents useful for erythropoiesis such as erythropoietin; antiulcer or antireflux agents such as famotidine, cimetidine, ranitidine hydrochloride; antinauseants or antiemetics such as meclizine hydrochloride, nabilone, prochlorperazine, dimenhydrinate, promethazine hydrochloride, thiethylperazine, scopolamine; oil-soluble vitamins such as vitamins A, D, E, K; and as well as other drugs such as mitotane, visadine, halonitrosoureas, anthrocyclines, ellipticine; or a combination thereof.

Bioactive agents can include chemotherapy drugs such as Abemaciclib, Abiraterone Acetate, Abitrexate (Methotrexate), Abraxane (Paclitaxel Albumin-stabilized Nanoparticle Formulation), Acalabrutinib, Adcetris (Brentuximab Vedotin), Ado-Trastuzumab Emtansine, Adriamycin (Doxorubicin Hydrochloride), Afatinib Dimaleate, Afinitor (Everolimus), Akynzeo (Netupitant and Palonosetron Hydrochloride), Aldara (Imiquimod), Aldesleukin, Alecensa (Alectinib), Alectinib, Alemtuzumab, Alimta (Pemetrexed Disodium), Aliqopa (Copanlisib Hydrochloride), Alkeran for Injection (Melphalan Hydrochloride), Alkeran Tablets (Melphalan), Aloxi (Palonosetron Hydrochloride), Alunbrig (Brigatinib), Ambochlorin (Chlorambucil), Amboclorin (Chlorambucil), Amifostine, Aminolevulinic Acid, Anastrozole, Aprepitant, Aredia (Pamidronate Disodium), Arimidex (Anastrozole), Aromasin (Exemestane), Arranon (Nelarabine), Arsenic Trioxide, Arzerra (Ofatumumab), Asparaginase Erwinia chrysanthemi, Atezolizumab, Avastin (Bevacizumab), Avelumab, Axicabtagene Ciloleucel, Axitinib, Azacitidine, Bavencio (Avelumab), Becenum (Carmustine), Beleodaq (Belinostat), Belinostat, Bendamustine Hydrochloride, Besponsa (Inotuzumab Ozogamicin), Bevacizumab, Bexarotene, Bexxar (Tositumomab and Iodine I 131 Tositumomab), Bicalutamide, BiCNU (Carmustine), Bleomycin, Blinatumomab, Blincyto (Blinatumomab), Bortezomib, Bosulif (Bosutinib), Bosutinib, Brentuximab Vedotin, Brigatinib, BuMel, Busulfan, Busulfex (Busulfan), Cabazitaxel, Cabometyx (Cabozantinib-S-Malate), Cabozantinib-S-Malate, Calquence (Acalabrutinib), Campath (Alemtuzumab), Camptosar (Irinotecan Hydrochloride), Capecitabine, Carac (Fluorouracil—Topical), Carboplatin, CARBOPLATIN-TAXOL, Carfilzomib, Carmubris (Carmustine), Carmustine, Carmustine Implant, Casodex (Bicalutamide), Ceritinib, Cerubidine (Daunorubicin Hydrochloride), Cervarix (Recombinant HPV Bivalent Vaccine), Cetuximab, CEV, Chlorambucil, CHLORAMBUCIL-PREDNISONE, Cisplatin, Cladribine, Clafen (Cyclophosphamide), Clofarabine, Clofarex (Clofarabine), Clolar (Clofarabine), CMF, Cobimetinib, Cometriq (Cabozantinib-S-Malate), Copanlisib Hydrochloride, Cosmegen (Dactinomycin), Cotellic (Cobimetinib), Crizotinib, Cyclophosphamide, Cyfos (Ifosfamide), Cyramza (Ramucirumab), Cytarabine, Cytarabine Liposome, Cytosar-U (Cytarabine), Cytoxan (Cyclophosphamide), Dabrafenib, Dacarbazine, Dacogen (Decitabine), Dactinomycin, Daratumumab, Darzalex (Daratumumab), Dasatinib, Daunorubicin Hydrochloride, Daunorubicin Hydrochloride and Cytarabine Liposome, Decitabine, Defibrotide Sodium, Defitelio (Defibrotide Sodium), Degarelix, Denileukin Diftitox, Denosumab, DepoCyt (Cytarabine Liposome), Dexamethasone, Dexrazoxane Hydrochloride, Dinutuximab, Docetaxel, Doxil (Doxorubicin Hydrochloride Liposome), Doxorubicin Hydrochloride, Doxorubicin Hydrochloride Liposome, Dox-SL (Doxorubicin Hydrochloride Liposome), DTIC-Dome (Dacarbazine), Durvalumab, Efudex (Fluorouracil—Topical), Elitek (Rasburicase), Ellence (Epirubicin Hydrochloride), Elotuzumab, Eloxatin (Oxaliplatin), Eltrombopag Olamine, Emend (Aprepitant), Empliciti (Elotuzumab), Enasidenib Mesylate, Enzalutamide, Epirubicin Hydrochloride, Erbitux (Cetuximab), Eribulin Mesylate, Erivedge (Vismodegib), Erlotinib Hydrochloride, Erwinaze (Asparaginase Erwinia chrysanthemi), Ethyol (Amifostine), Etopophos (Etoposide Phosphate), Etoposide, Etoposide Phosphate, Evacet (Doxorubicin Hydrochloride Liposome), Everolimus, Evista (Raloxifene Hydrochloride), Evomela (Melphalan Hydrochloride), Exemestane, 5-FU (Fluorouracil Injection), 5-FU (Fluorouracil-Topical), Fareston (Toremifene), Farydak (Panobinostat), Faslodex (Fulvestrant), FEC, Femara (Letrozole), Filgrastim, Fludara (Fludarabine Phosphate), Fludarabine Phosphate, Fluoroplex (Fluorouracil—Topical), Fluorouracil Injection, Fluorouracil—Topical, Flutamide, Folex (Methotrexate), Folex PFS (Methotrexate), FOLFIRI, FOLFIRI-BEVACIZUMAB, FOLFIRI-CETUXIMAB, FOLFIRINOX, FOLFOX, Folotyn (Pralatrexate), FU-LV, Fulvestrant, Gardasil (Recombinant HPV Quadrivalent Vaccine), Gardasil 9 (Recombinant HPV Nonavalent Vaccine), Gazyva (Obinutuzumab), Gefitinib, Gemcitabine Hydrochloride, GEMCITABINE-CISPLATIN, GEMCITABINE-OXALIPLATIN, Gemtuzumab Ozogamicin, Gemzar (Gemcitabine Hydrochloride), Gilotrif (Afatinib Dimaleate), Gleevec (Imatinib Mesylate), Gliadel (Carmustine Implant), Gliadel wafer (Carmustine Implant), Glucarpidase, Goserelin Acetate, Halaven (Eribulin Mesylate), Hemangeol (Propranolol Hydrochloride), Herceptin (Trastuzumab), HPV Bivalent Vaccine, Recombinant, HPV Nonavalent Vaccine, Recombinant, HPV Quadrivalent Vaccine, Recombinant, Hycamtin (Topotecan Hydrochloride), Hydrea (Hydroxyurea), Hydroxyurea, Hyper-CVAD, Ibrance (Palbociclib), Ibritumomab Tiuxetan, Ibrutinib, ICE, Iclusig (Ponatinib Hydrochloride), Idamycin (Idarubicin Hydrochloride), Idarubicin Hydrochloride, Idelalisib, Idhifa (Enasidenib Mesylate), Ifex (Ifosfamide), Ifosfamide, Ifosfamidum (Ifosfamide), IL-2 (Aldesleukin), Imatinib Mesylate, Imbruvica (Ibrutinib), Imfinzi (Durvalumab), Imiquimod, Imlygic (Talimogene Laherparepvec), Inlyta (Axitinib), Inotuzumab Ozogamicin, Interferon Alfa-2b, Recombinant, Interleukin-2 (Aldesleukin), Intron A (Recombinant Interferon Alfa-2b), Iodine I 131 Tositumomab and Tositumomab, Ipilimumab, Iressa (Gefitinib), Irinotecan Hydrochloride, Irinotecan Hydrochloride Liposome, Istodax (Romidepsin), Ixabepilone, Ixazomib Citrate, Ixempra (Ixabepilone), Jakafi (Ruxolitinib Phosphate), Jevtana (Cabazitaxel), Kadcyla (Ado-Trastuzumab Emtansine), Keoxifene (Raloxifene Hydrochloride), Kepivance (Palifermin), Keytruda (Pembrolizumab), Kisqali (Ribociclib), Kymriah (Tisagenlecleucel), Kyprolis (Carfilzomib), Lanreotide Acetate, Lapatinib Ditosylate, Lartruvo (Olaratumab), Lenalidomide, Lenvatinib Mesylate, Lenvima (Lenvatinib Mesylate), Letrozole, Leucovorin Calcium, Leukeran (Chlorambucil), Leuprolide Acetate, Leustatin (Cladribine), Levulan (Aminolevulinic Acid), Linfolizin (Chlorambucil), LipoDox (Doxorubicin Hydrochloride Liposome), Lomustine, Lonsurf (Trifluridine and Tipiracil Hydrochloride), Lupron (Leuprolide Acetate), Lupron Depot (Leuprolide Acetate), Lupron Depot-Ped (Leuprolide Acetate), Lynparza (Olaparib), Marqibo (Vincristine Sulfate Liposome), Matulane (Procarbazine Hydrochloride), Mechlorethamine Hydrochloride, Megestrol Acetate, Mekinist (Trametinib), Melphalan, Melphalan Hydrochloride, Mercaptopurine, Mesna, Mesnex (Mesna), Methazolastone (Temozolomide), Methotrexate, Methotrexate LPF (Methotrexate), Methylnaltrexone Bromide, Mexate (Methotrexate), Mexate-AQ (Methotrexate), Midostaurin, Mitomycin C, Mitoxantrone Hydrochloride, Mitozytrex (Mitomycin C), Mozobil (Plerixafor), Mustargen (Mechlorethamine Hydrochloride), Mutamycin (Mitomycin C), Myleran (Busulfan), Mylosar (Azacitidine), Mylotarg (Gemtuzumab Ozogamicin), Nanoparticle Paclitaxel (Paclitaxel Albumin-stabilized Nanoparticle Formulation), Navelbine (Vinorelbine Tartrate), Necitumumab, Nelarabine, Neosar (Cyclophosphamide), Neratinib Maleate, Nerlynx (Neratinib Maleate), Netupitant and Palonosetron Hydrochloride, Neulasta (Pegfilgrastim), Neupogen (Filgrastim), Nexavar (Sorafenib Tosylate), Nilandron (Nilutamide), Nilotinib, Nilutamide, Ninlaro (Ixazomib Citrate), Niraparib Tosylate Monohydrate, Nivolumab, Nolvadex (Tamoxifen Citrate), Nplate (Romiplostim), Obinutuzumab, Odomzo (Sonidegib), OEPA, Ofatumumab, OFF, Olaparib, Olaratumab, Omacetaxine Mepesuccinate, Oncaspar (Pegaspargase), Ondansetron Hydrochloride, Onivyde (Irinotecan Hydrochloride Liposome), Ontak (Denileukin Diftitox), Opdivo (Nivolumab), Osimertinib, Oxaliplatin, Paclitaxel, Paclitaxel Albumin-stabilized Nanoparticle Formulation, Palbociclib, Palifermin, Palonosetron Hydrochloride, Palonosetron Hydrochloride and Netupitant, Pamidronate Disodium, Panitumumab, Panobinostat, Paraplat (Carboplatin), Paraplatin (Carboplatin), Pazopanib Hydrochloride, Pegaspargase, Pegfilgrastim, Peginterferon Alfa-2b, PEG-Intron (Peginterferon Alfa-2b), Pembrolizumab, Pemetrexed Disodium, Perjeta (Pertuzumab), Pertuzumab, Platinol (Cisplatin), Platinol-AQ (Cisplatin), Plerixafor, Pomalidomide, Pomalyst (Pomalidomide), Ponatinib Hydrochloride, Portrazza (Necitumumab), Pralatrexate, Prednisone, Procarbazine Hydrochloride, Proleukin (Aldesleukin), Prolia (Denosumab), Promacta (Eltrombopag Olamine), Propranolol Hydrochloride, Provenge (Sipuleucel-T), Purinethol (Mercaptopurine), Purixan (Mercaptopurine), Radium 223 Dichloride, Raloxifene Hydrochloride, Ramucirumab, Rasburicase, Recombinant Human Papillomavirus (HPV) Bivalent Vaccine, Recombinant Human Papillomavirus (HPV) Nonavalent Vaccine, Recombinant Human Papillomavirus (HPV) Quadrivalent Vaccine, Recombinant Interferon Alfa-2b, Regorafenib, Relistor (Methylnaltrexone Bromide), Revlimid (Lenalidomide), Rheumatrex (Methotrexate), Ribociclib, Rituxan (Rituximab), Rituxan Hycela (Rituximab and Hyaluronidase Human), Rituximab, Rituximab and Hyaluronidase Human, Rolapitant Hydrochloride, Romidepsin, Romiplostim, Rubidomycin (Daunorubicin Hydrochloride), Rubraca (Rucaparib Camsylate), Rucaparib Camsylate, Ruxolitinib Phosphate, Rydapt (Midostaurin), Sclerosol Intrapleural Aerosol (Talc), Siltuximab, Sipuleucel-T, Somatuline Depot (Lanreotide Acetate), Sonidegib, Sorafenib Tosylate, Sprycel (Dasatinib), STANFORD V, Sterile Talc Powder (Talc), Steritalc (Talc), Stivarga (Regorafenib), Sunitinib Malate, Sutent (Sunitinib Malate), Sylatron (Peginterferon Alfa-2b), Sylvant (Siltuximab), Synribo (Omacetaxine Mepesuccinate), Tabloid (Thioguanine), Tafinlar (Dabrafenib), Tagrisso (Osimertinib), Talc, Talimogene Laherparepvec, Tamoxifen Citrate, Tarabine PFS (Cytarabine), Tarceva (Erlotinib Hydrochloride), Targretin (Bexarotene), Tasigna (Nilotinib), Taxol (Paclitaxel), Taxotere (Docetaxel), Tecentriq (Atezolizumab), Temodar (Temozolomide), Temozolomide, Temsirolimus, Thalidomide, Thalomid (Thalidomide), Thioguanine, Thiotepa, Tisagenlecleucel, Tolak (Fluorouracil—Topical), Topotecan Hydrochloride, Toremifene, Torisel (Temsirolimus), Tositumomab and Iodine I 131 Tositumomab, Totect (Dexrazoxane Hydrochloride), TPF, Trabectedin, Trametinib, Trastuzumab, Treanda (Bendamustine Hydrochloride), Trifluridine and Tipiracil Hydrochloride, Trisenox (Arsenic Trioxide), Tykerb (Lapatinib Ditosylate), Unituxin (Dinutuximab), Uridine Triacetate, VAC, Valrubicin, Valstar (Valrubicin), Vandetanib, Varubi (Rolapitant Hydrochloride), Vectibix (Panitumumab), Velban (Vinblastine Sulfate), Velcade (Bortezomib), Velsar (Vinblastine Sulfate), Vemurafenib, Venclexta (Venetoclax), Venetoclax, Verzenio (Abemaciclib), Viadur (Leuprolide Acetate), Vidaza (Azacitidine), Vinblastine Sulfate, Vincasar PFS (Vincristine Sulfate), Vincristine Sulfate, Vincristine Sulfate Liposome, Vinorelbine Tartrate, VIP (vasoactive intestinal peptide or vasoactive intestinal polypeptide), Vismodegib, Vistogard (Uridine Triacetate), Voraxaze (Glucarpidase), Vorinostat, Votrient (Pazopanib Hydrochloride), Vyxeos (Daunorubicin Hydrochloride and Cytarabine Liposome), Wellcovorin (Leucovorin Calcium), Xalkori (Crizotinib), Xeloda (Capecitabine), XELIRI, XELOX, Xgeva (Denosumab), Xofigo (Radium 223 Dichloride), Xtandi (Enzalutamide), Yervoy (Ipilimumab), Yescarta (Axicabtagene Ciloleucel), Yondelis (Trabectedin), Zaltrap (Ziv-Aflibercept), Zarxio (Filgrastim), Zejula (Niraparib Tosylate Monohydrate), Zelboraf (Vemurafenib), Zevalin (Ibritumomab Tiuxetan), Zinecard (Dexrazoxane Hydrochloride), Ziv-Aflibercept, Zofran (Ondansetron Hydrochloride), Zoladex (Goserelin Acetate), Zoledronic Acid, Zolinza (Vorinostat), Zometa (Zoledronic Acid), Zydelig (Idelalisib), Zykadia (Ceritinib), Zytiga (Abiraterone Acetate), or a combination thereof. If a bioactive agent comprises one or more of the functional groups that comprise C, N, O, P, S, an ether, an ester, an amide, a carbamate, a disulfide bond, a triazole, or a combination thereof, the LCFA can be linked to one or more of the functional groups of the bioactive agent. If the bioactive agent is free from one or more of the functional groups mentioned above, a linker, a spacer, or a combination thereof, can be added using the chemistry known to those in the field and the LCFA can be linked to the linker.

In some cases, the bioactive agent can comprise the poly-nucleic acid comprising a DNA, an RNA, a poly-oligonucleotide, a poly-oligodeoxynucleotide, a derivative thereof, or a combination thereof. In some cases, the long chain fatty acid (LCFA) can be linked to the poly-nucleic acid at a 3′-end nuclei acid residual of the poly-nucleic acid. As used herein, a poly-nucleic acid derivative thereof can comprise a part of the poly-nucleic acid, a fragment of the poly-nucleic acid, one or more modified nucleic acid residues, one or more substituted nucleic acid residues, a homolog poly-nucleic acid having at least 75% homology to the poly-nucleic acid, a modified poly-nucleic acid having substantially the same function as the poly-nucleic acid, or a combination thereof.

In some cases, each of the DNA, RNA, poly-oligonucleotide, poly-oligodeoxynucleotide, a derivative thereof, or a combination thereof, can be a single strand poly-nucleic acid, and wherein the pharmaceutical composition can further comprise a complementary strand of the poly-nucleic acid.

In some cases, the pharmaceutical composition disclosed herein can comprise siRNA, microRNAs, a derivative thereof, or a combination thereof.

In some cases, the pharmaceutical composition can comprise palmitate-conjugated microRNA-34a (miR-34a) mimic The pharmaceutical composition can further comprise the sense strand. A representative example of a structure of palmitate-conjugated microRNA-34a (miR-34a) mimic antisense strand annealed to a sense strand is shown as formulas 4 herein and in FIG. 17A. The antisense strand conjugated to palmitate through a linker without the sense strand is shown as formula 5 herein and in FIG. 17B. The nucleotides in the sense and antisense strands are matched based on A-U and G-C pairing. There can be a TT overhang at the 3′ end of both antisense and sense strands. A miR-34a derivative thereof can comprise a part of the miR-34a, a fragment of the miR-34a, one or more modified nucleic acid residues, one or more substituted nucleic acid residues, a homolog poly-nucleic acid having at least 75% homology to the miR-34a, a modified poly-nucleic acid having substantially the same function as the miR-34a, or a combination thereof.

In some cases, the pharmaceutical composition can comprise the bioactive agent and the long chain fatty acid (LCFA) having the

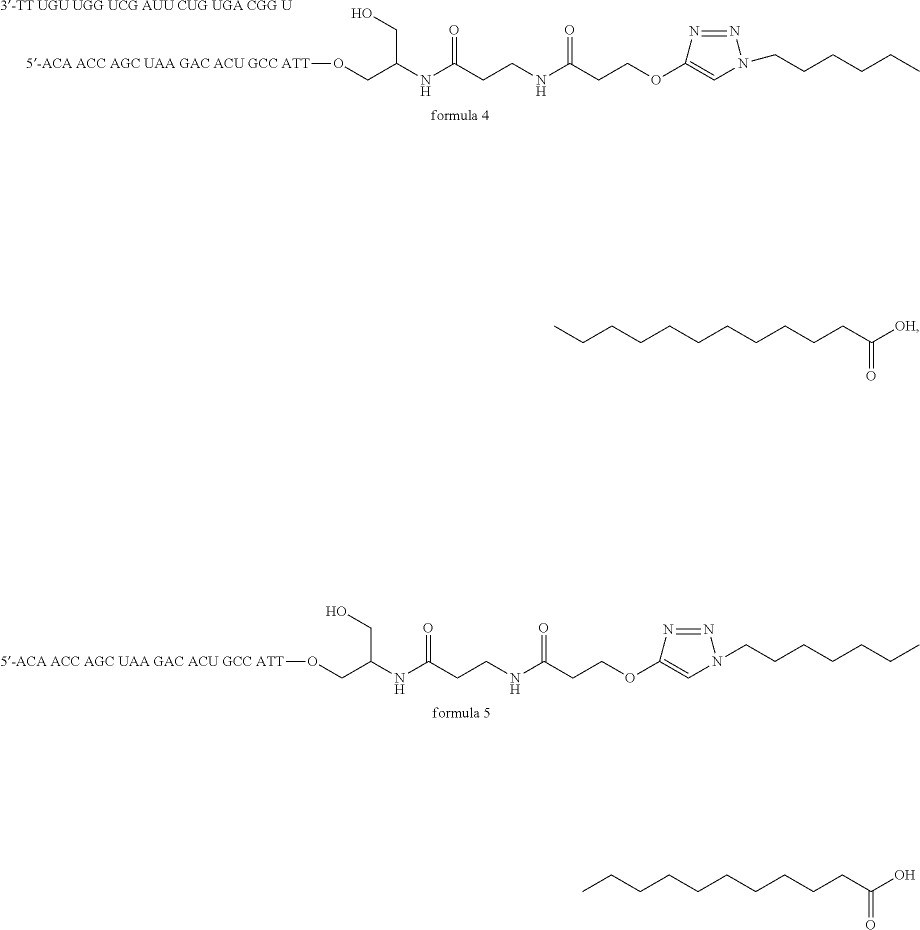

or a combination thereof.

In some cases, the siRNA and microRNA mimics can comprise Let-7 mimic for cancer therapy, miR-155 mimic to modulate immune responses, human IRE1 siRNA for immunomodulation and disease treatment, a derivative thereof, or a combination thereof.

In some cases, the bioactive agent can comprise Let-7 mimic There are many choices for Let-7 mimic In one example, the sense strand of Let-7 mimic can comprise: 5′-CUGUACAGCCUCCUAGCUUUCCTT-3′ (SEQ ID. 7). Chemical conjugation can be at the 3′ nucleotide. The pharmaceutical composition can comprise antisense Let-7 mimic-LCFA, sense Let-7 mimic-LCFA, an antisense Let-7 mimic-LCFA annealed with a sense Let-7 mimic strand, a sense Let-7 mimic-LCFA annealed with an antisense Let-7 mimic strand, or a combination thereof. A Let-7 mimic derivative thereof can comprise a part of the Let-7 mimic, a fragment of the Let-7 mimic, one or more modified nucleic acid residues, one or more substituted nucleic acid residues, a homolog poly-nucleic acid having at least 75% homology to the Let-7 mimic, a modified poly-nucleic acid having substantially the same function as the Let-7 mimic, or a combination thereof.

In some cases, the bioactive agent can comprise miR-155 mimic to modulate immune responses. There are many choices for miR-155 mimic In one example, the sense strand of miR-155 mimic can comprise:

5′-UUAAUGCUAAUUGUGAUAGGGGUTT-3′ (SEQ ID. 8). Chemical conjugation can be at the 3′ nucleotide. The pharmaceutical composition can comprise antisense miR-155 mimic-LCFA, sense miR-155 mimic-LCFA, an antisense miR-155 mimic-LCFA annealed with a sense miR-155 mimic strand, a sense miR-155 mimic-LCFA annealed with an antisense miR-155 mimic strand, or a combination thereof. A miR-155 mimic derivative thereof can comprise a part of the miR-155 mimic, a fragment of the miR-155 mimic, one or more modified nucleic acid residues, one or more substituted nucleic acid residues, a homolog poly-nucleic acid having at least 75% homology to the miR-155 mimic, a modified poly-nucleic acid having substantially the same function as the miR-155 mimic, or a combination thereof.

In some cases, the bioactive agent can comprise human IRE1 siRNA for immunomodulation and disease treatment. There are many choices for IRE1 siRNA. In one example, the sense strand of human IRE1 siRNA can comprise:

5′-GCACGUGAAUUGAUAGAGATT-3′ (SEQ ID. 9). Chemical conjugation can be at the 3′ nucleotide. The pharmaceutical composition can comprise antisense human IRE1 siRNA-LCFA, sense human IRE1 siRNA-LCFA, an antisense human IRE1 siRNA-LCFA annealed with a sense human IRE1 siRNA strand, a sense human IRE1 siRNA-LCFA annealed with an antisense human IRE1 siRNA strand, or a combination thereof. A human IRE1 siRNA derivative thereof can comprise a part of the human IRE1 siRNA, a fragment of the human IRE1 siRNA, one or more modified nucleic acid residues, one or more substituted nucleic acid residues, a homolog poly-nucleic acid having at least 75% homology to the human IRE1 siRNA, a modified poly-nucleic acid having substantially the same function as the human IRE1 siRNA, or a combination thereof.

The well known “Click Chemistry” (Kolb, H. C.; Angewandte Chemie International Edition. 40 (11):2004-2021, 2001) can be used to link the long chain fatty acid to the DNA, RNA, poly-oligonucleotide, poly-oligodeoxynucleotide, derivative thereof, or a combination thereof.

In some cases, the pharmaceutical composition can comprise one bioactive agent linked to two or more LCFAs, wherein the two or more LCFA can be the same or different. In one example, the pharmaceutical composition can comprise the formula 1 (FIG. 6A). In some cases, the pharmaceutical composition can comprise one bioactive agent linked to two or more LCFAs, wherein the two or more LCFA can comprise one or more linear or branched saturated C10 to C40 fatty acids, one or more linear or branched unsaturated C10 to C40 fatty acids, or a combination thereof.

The pharmaceutical composition of this disclosure can further comprise a pharmaceutically acceptable excipient selected from a detergent, a buffer, a phosphate, a salt, a water, a solvent, a filler, an inorganic compound, an organic compound, a synthetic polymer, a biopolymer, a sugar, a starch, a polysaccharide, SiO₂, Eudragit L-100-coated SiO₂, or a combination thereof. Other excipients disclosed herein, such as those listed in the US FDA inactive ingredient database (IID) or reagents determined to be generally regarded as safe (GRAS), or a combination thereof, can also be suitable.

The pharmaceutical composition can be formulated as an enteric-coated capsule, an enteric-coated tablet, an enteric-coated drug particle suspension, an enteric-coated drug powder, or a combination thereof. In some cases, each of the enteric-coated capsule, the enteric-coated tablet, the enteric-coated drug particle suspension, the enteric-coated drug powder, or a combination thereof, can comprise hard gelatin.

Enteric-coated formulations can protect a bioactive agent (also referred to as a therapeutic agent, and can be used interchangeably herein) from attack by acidic fluid and digestive enzymes in the GI track and can release the bioactive agent in the pH-neutral small intestine where the bioactive agent can be absorbed by the intestinal epithelial cells. The LCFA-drug conjugates can be either packaged in acid-resistant capsules or mixed with excipients before they are coated with pH-responsive polymers. The drug conjugates are protected in the acidic gastric fluid. Once in a neutral pH environment, as in the lumen of jejunum or ileum, the pH-sensitive coating dissociates from the drug capsule or other forms of drug packages, resulting in release of the therapeutic agent. Some examples of pH-sensitive polymers can comprise anionic polymers, such as P(MAA-g-EG), P(IA-co-NVP), P(MAA-co-NVP), Alginate-based polymer, hyaluronic acid-based polymer, cationic polymer, such as chitosan-based polymer, amphiphilic polymer, such as P(MAA-g-EG) with PMMA nanoparticles, degrading polymers, such as dextran-based polymer, gelatin-based polymer, carboxymethyl cellulose or poly(acrylic acid) hydrogels, maleic acid cross linked poly(vinyl alcohol), azoaromatic crosslinked polymers, BC-g-P(AA), guar gum-poly(acrylic acid)-(-cyclodextrin)(GG-PAA-CD) (Liu, L.; et al., Drug Deliv, 24(1):569-581, 2017). Other polymers can also be suitable. The polymers that can deliver an API in intestine can be preferred.

In some cases, this disclosure is directed to a process for producing a pharmaceutical composition comprising a bioactive agent covalently conjugated to at least one long chain fatty acid (LCFA), the process comprising:

a) providing a long chain dicarboxylic acid comprising a carbon chain having at least 10 carbon atoms, a first chain end having a first acid group and a second chain end having a second acid group, the first acid group and the second acid group are connected by the carbon chain;

b) reacting the first acid group, optionally via a linker, with the bioactive agent to produce the pharmaceutical composition;

wherein the second chain end in the pharmaceutical composition comprises a free carboxylic acid group or a salt thereof.

A long chain dicarboxylic acid form of any of the LCFAs disclosed herein can be suitable.

The bioactive agent can be selected from a polypeptide, a small molecule drug, a poly-nucleic acid, or a combination thereof, wherein the long chain dicarboxylic acid can be selected from a C10 to C40 branched or linear saturated dicarboxylic acid, a C10 to C40 branched or linear unsaturated dicarboxylic acid, or a combination thereof, and wherein the long chain dicarboxylic acid can be linked to the bioactive agent via one or more functional groups that can comprise C, N, O, P, S, an ether, an ester, an amide, a carbamate, a disulfide bond, a triazole, or a combination thereof, wherein the functional groups can be on the bioactive agent, on the linker when present, or a combination thereof, and wherein the linker can comprise 0 to 40 atoms.

A dicarboxylic acid form of any of the long chain fatty acids disclosed above can be suitable. In some cases, a long chain dicarboxylic acid can be selected from nonanedioic acid (a C9 saturated dicarboxylic), devanedioic acid (C10), undecanedioic acid (C11), dodecanedioic acid (C12), tridecanedioic acid (C13), hexadecanedioic acid (C16), octadecanedioic acid (C18), decosanedioic acid (C22), or a combination thereof. The dicarboxylic can be reacted to the bioactive agent, such as a protein or polypeptide under reaction condition suitable for amine and carboxylic acid reactions. In some cases, a dicarboxylic acid can be reacted with N-hydroxysuccinimide (NHS) in the presence of N-(3-Dimethylaminopropyl)-N′-ethylcarbodiimide hydrochloride (EDC) in a first reaction step, and then react with a polypeptide in presence of N,N-Diisopropylethylamine (DIPEA) in the second step. The reaction can be stopped by adding 1% trifluoro acetic acid (TFA) to the reaction mixture.

In some cases, the bioactive agent can comprise a polypeptide and wherein the linker can comprise 0 to 10 atoms and the linker can be free from glutamic acid, glutamic acid derivative, glutamine, glutamine derivative, or a combination thereof.

In some cases, the polypeptide can comprise amino acid, modified amino acid, amino acid analogue, or a combination thereof, and the long chain fatty acid (LCFA) can be linked to the polypeptide at one or more —N—, —NH—, —NH₂, —S—, —SH, —OH, —COO— or a combination thereof, of one or more residues of the amino acid, modified amino acid, amino acid analogue, or a combination thereof, of the polypeptide, via one of the carboxylic acid groups of the dicarboxylic acid.

In some cases, the bioactive agent can comprise a glucagon or a derivative thereof, a glucagon analogue or a derivative thereof, a glucagon-like peptide 1 (GLP-1) or a derivative thereof, exendin-4 or a derivative thereof, insulin or a derivative thereof, human brain natriuretic peptide or a derivative thereof, octreotide or a derivative thereof, human vasoactive intestinal peptide (VIP) or a derivative thereof, or a combination thereof.

In some cases, the pharmaceutical composition produced by the process can comprise the bioactive agent and the long chain fatty acid (LCFA) having the

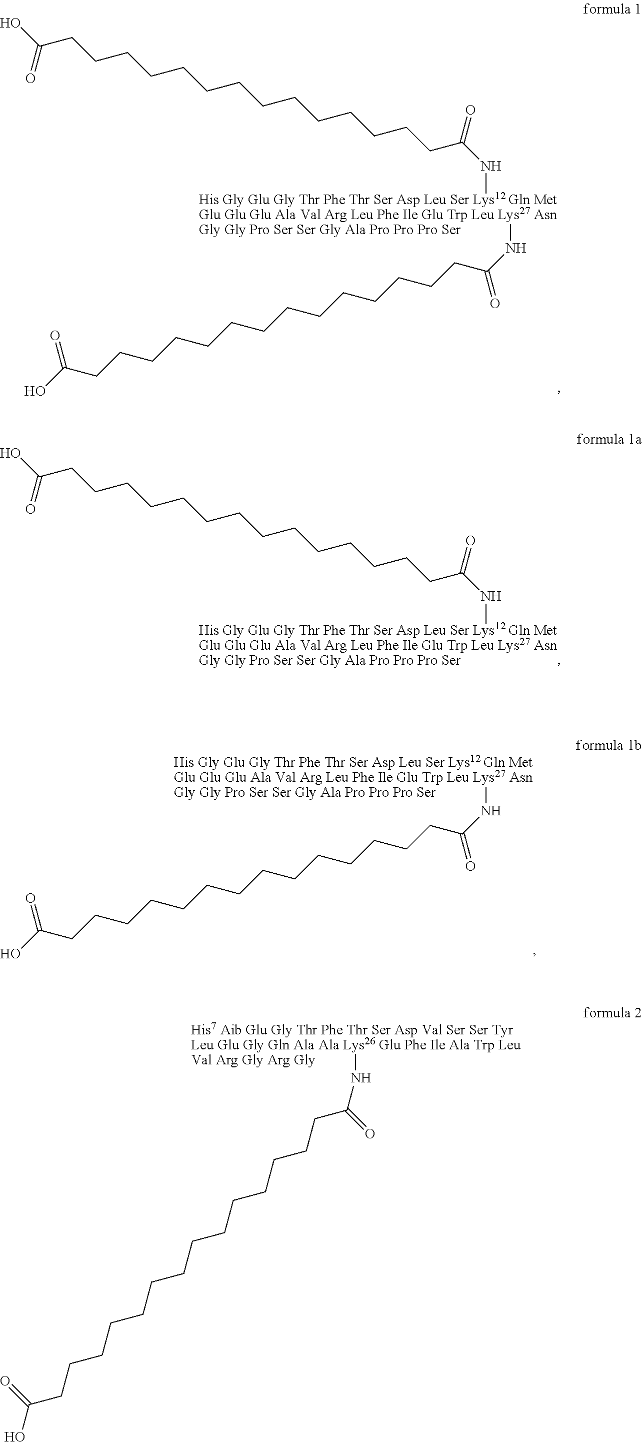

or a combination thereof.

In some cases, the pharmaceutical compositions of this disclosure can comprise a glucagon-like peptide 1 (GLP-1) linked to a LCFA at Lys²⁶ (GLP-1-Lys²⁶-LCFA). The GLP-1 can be linked to the LCFA via a linker that can comprise 0 to 10 atoms and the linker can be free from glutamic acid, glutamic acid derivative, glutamine, glutamine derivative, or a combination thereof. In some cases, the pharmaceutical composition can comprise a GLP-1 linked directly to the LCFA via an amino group of Lys²⁶ without a linker, wherein the long chain fatty acid (LCFA) can comprise a C10 to C40 branched or linear saturated fatty acid, C10 to C40 branched or linear unsaturated fatty acid, or a combination thereof.

In some cases, the pharmaceutical compositions of this disclosure can comprise an exendin-4 (Ex4) linked to a LCFA at Lys¹² (Ex4-Lys¹²-LCFA), an exendin-4 (Ex4) linked to a LCFA at Lys²⁷ (Ex4-Lys²⁷-LCFA), an exendin-4 (Ex4) linked to a LCFA at both Lys¹² and Lys²⁷ (Ex4-Lys¹²/Lys²⁷-LCFA), or a combination thereof. The Ex4 can be linked to the LCFA via the linker that can comprise 0 to 10 atoms and the linker can be free from glutamic acid, glutamic acid derivative, glutamine, glutamine derivative, or a combination thereof. In some cases, the pharmaceutical composition can comprise Ex4 directly to the LCFA via an amino group of Lys¹², an amino group of Lys²⁷, both amino groups of Lys¹² and Lys²⁷, without a linker, wherein the long chain fatty acid (LCFA) can comprise a C10 to C40 branched or linear saturated fatty acid, C10 to C40 branched or linear unsaturated fatty acid, or a combination thereof.

In some cases, the bioactive agent can comprise exendin-4 (Ex4) (SEQ ID. 1). An example of representative Ex4-LCFA is shown as Ex4-Lys¹²&²⁷-LCFA in FIG. 6A. Some examples of measurement data on Ex4 (free Ex4), Ex4-Lys¹²&²⁷-LCFA, Ex4-Lys²⁷-LCFA, Ex4-Lys¹²-LCFA are shown in FIG. 7, FIG. 8, FIG. 9, FIG. 10 and FIG. 11.

In some cases, the bioactive agent can comprise glucagon-like peptide 1 (GLP-1) or a GLP-1 derivative with modified amino acid Aib (SEQ ID. 2). An example of representative GLP-1-LCFA is shown as GLP-1-Lys²⁶-LCFA (FIG. 6B). The LCFA can be linked to the lysine residue as the 26th amino acid position. Some examples of measurement data on GLP-1 (free GLP-1), GLP-1-Lys²⁶-LCFA, GLP-1-Lys²⁶-LCFA capsule, and dosage effects are shown in FIG. 12, FIG. 13 and FIG. 14.

In some cases, the bioactive agent can comprise human brain natriuretic peptide for heart failure treatment. The human brain natriuretic peptide can have an amino acid sequence: Ser Pro Lys Met Val Gln Gly Ser Gly Cys Phe Gly Arg Lys Met Asp Arg Ile Ser Ser Ser Ser Gly Leu Gly Cys Lys Val Leu Arg Arg His (SEQ ID. 5). There can be a disulfide bond between the two Cys residues.

In some cases, the bioactive agent can comprise octreotide for cancer treatment. The octreotide can have an amino acid sequence: D-Phe Cys Phe D-Trp Lys Thr Cys Thr (SEQ ID. 6). There can be D-Phe and D-Trp amino acid residues in the peptide. There can be a disulfide bond between the two Cys residues.

In some cases, the bioactive agent can comprise human vasoactive intestinal peptide (VIP) for restoring organ function. A representative human VIP can have a peptide sequence: His Ser Asp Ala Val Phe Thr Asp Asn Tyr Thr Arg Leu Arg Lys Gln Met Ala Val Lys Lys Tyr Leu Asn Ser Ile Leu Asn (SEQ ID. 10). Chemical conjugation of the VIP and LCFA can be linked at one or more of the Lys residues, the N-terminal —NH₂ group, the C-termina —COOH group, or a combination thereof.

In some cases, the bioactive agent can comprise an ibrutinib. Examples of chemical structures of ibrutinib, LCFA-ibrutinib and measurement data are shown in FIG. 15A, FIG. 15B, FIG. 15C, FIG. 16A and FIG. 16B, respectively.

In some cases, the bioactive agent can comprise a metabolic drug, a cancer drug, a chemotherapeutic drug, analgesics/antipyretics, anesthetics, antiasthmatics, antidepressants, antidiabetics, antifungal agents antihypertensive agents, antipsychotic agents, antimanic agents, antiarrhythmics, antiarthritic agents, antigout agents, anticoagulants, thrombolytic agents, antifibrinolytic agents, hemorheologic agents, antiplatelet agents, anticonvulsants, antiparkinson agents, antihistamines/antipruritics, agents useful for calcium regulation, antibacterial agents, antiviral agents, antimicrobials, antibiotics, anti-infectives, corticosteroids, thyroid hormones, hypoglycemic agents selected from insulin, recombinant insulin, glyburide, chlorpropamide, glipizide, tolbutamide, tolazamide, or a combination thereof, hypolipidemic agents selected from clofibrate, dextrothyroxine sodium, probucol, lovastatin, niacin or a combination thereof, antiulcer or antireflux agents, antinauseants or antiemetics, vitamins, mitotane, visadine, halonitrosoureas, anthrocyclines, ellipticine, or a combination thereof. Other bioactive agents disclosed above can also be suitable.

In some cases, the pharmaceutical composition produced by the process can comprise the bioactive agent and the long chain fatty acid (LCFA) having the

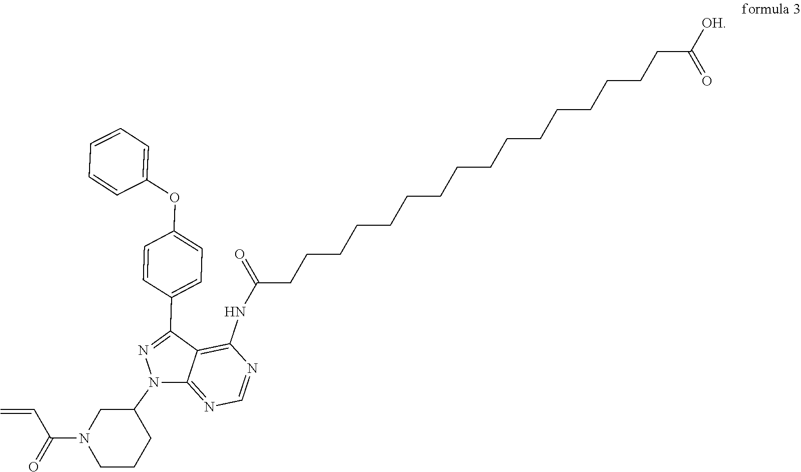 [SMILES: C=CC(=O)N1CCCC(n2nc(-c3ccc(Oc4ccccc4)cc3)c3c(NC(=O)CCCCCCCCCCCCCCCCC(=O)O)ncnc32)C1]

In some cases, the bioactive agent can comprise the poly-nucleic acid comprising a DNA, an RNA, a poly-oligonucleotide, a poly-oligodeoxynucleotide, a derivative thereof, or a combination thereof.

In some cases, the long chain fatty acid (LCFA) can be linked to the poly-nucleic acid at a 3′-end nuclei acid residual of the poly-nucleic acid.

In some cases, each of the DNA, RNA, poly-oligonucleotide, poly-oligodeoxynucleotide, derivative thereof, or a combination thereof, can be a single strand poly-nucleic acid, and wherein the pharmaceutical composition produced by the process can further comprise a complementary strand of the poly-nucleic acid.

Aforementioned “Click Chemistry” can be used to link the fatty acid to the DNA, RNA, poly-oligonucleotide, poly-oligodeoxynucleotide, derivative thereof, or a combination thereof.

In some cases, the pharmaceutical composition comprises the bioactive agent and the long chain fatty acid (LCFA) having the

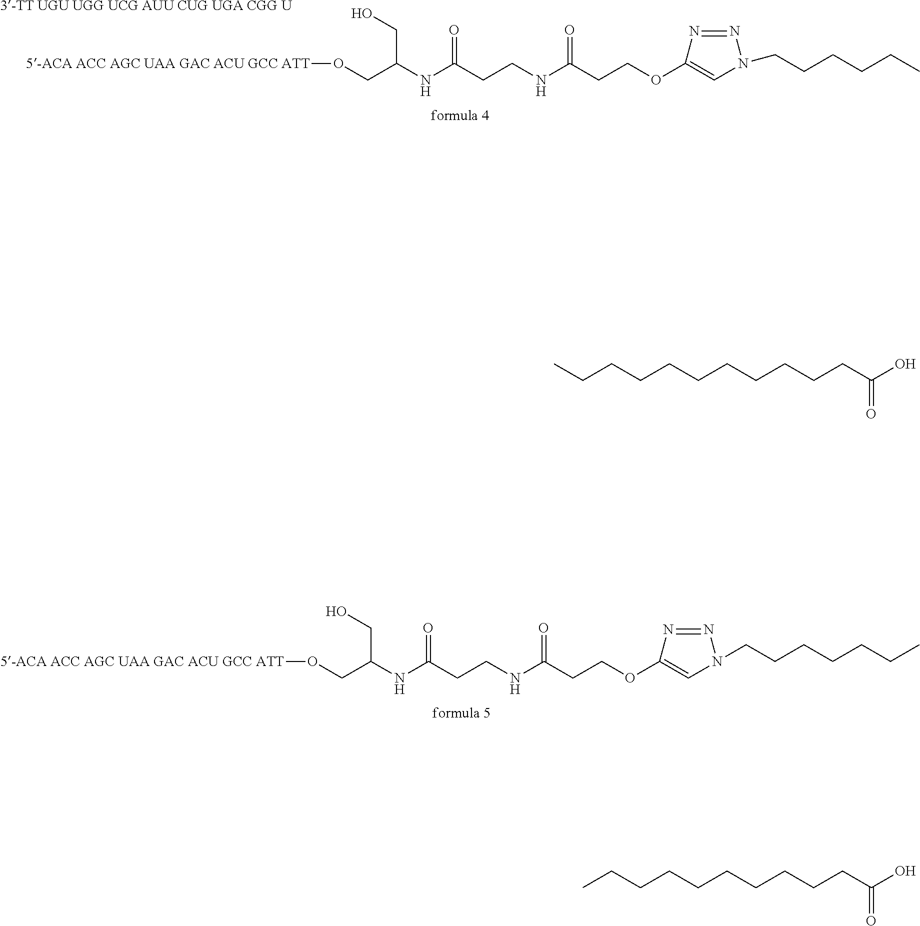

or a combination thereof.

The process of this disclosure can further comprise mixing a pharmaceutically acceptable excipient selected from a detergent, a buffer, a phosphate, a salt, a water, a solvent, a filler, an inorganic compound, an organic compound, a synthetic polymer, a biopolymer, a sugar, a starch, a polysaccharide, SiO₂, Eudragit L-100-coated SiO₂, or a combination thereof, with the bioactive agent and the long chain fatty acid (LCFA).

The process can further comprise formulating the pharmaceutical composition as an enteric-coated capsule, an enteric-coated tablet, an enteric-coated drug particle suspension, an enteric-coated drug powder, or a combination thereof. In some cases, the process can comprise mixing the bioactive agent-LCFA with an enteric agent comprising hard gelatin to produce the enteric-coated capsule, the enteric-coated tablet, the enteric-coated drug particle suspension, the enteric-coated drug powder, or a combination thereof.

In some cases, the process can comprise mixing the LCFA-drug conjugates in acid-resistant capsules or mixed with excipients before they are coated with pH-responsive polymers.

In some cases, pH-sensitive polymers can be selected from anionic polymers, such as P(MAA-g-EG), P(IA-co-NVP), P(MAA-co-NVP), Alginate-based polymer, hyaluronic acid-based polymer, cationic polymer, such as chitosan-based polymer, amphiphilic polymer, such as P(MAA-g-EG) with PMMA nanoparticles, degrading polymers, such as dextran-based polymer, gelatin-based polymer, carboxymethyl cellulose or poly(acrylic acid) hydrogels, maleic acid cross linked poly(vinyl alcohol), azoaromatic crosslinked polymers, BC-g-P(AA), guar gum-poly(acrylic acid)-(-cyclodextrin)(GG-PAA-CD) (Liu, L.; et al., Drug Deliv, 24(1):569-581, 2017). Other polymers can also be suitable. The polymers that can deliver an API in intestine can be preferred.

In some cases, the disclosure is also directed to a method for treating a disease of a subject in need thereof, the method comprises orally administering a pharmaceutically effective dosage of any of the pharmaceutical compositions disclosed herein to the subject.

The disease can be a metabolic disorder, a cancer, respiratory disease, depressing, diabetics, infectious disease, hypertensive, allergy, psychotic disorder, neurological disorder, arthritic disease, coagulation disease, ulcer, reflux, nausea, vitamins deficiency, heart failure, immunodeficiency, or a combination thereof.

In some cases, this disclosure is directed to a use of a composition comprising a bioactive agent covalently conjugated to at least one long chain fatty acid (LCFA) for manufacturing a medicament for treating a disease, wherein the long chain fatty acid can comprise a carbon chain having at least 10 carbon atoms, a first chain end and a second chain end connected by said carbon chain, wherein the first chain end can be linked to the bioactive agent and the second chain end can comprise a free carboxylic acid group, and wherein the disease can be a metabolic disorder, a cancer, respiratory disease, depressing, diabetics, infectious disease, hypertensive, allergy, psychotic disorder, neurological disorder, arthritic disease, coagulation disease, ulcer, reflux, nausea, vitamins deficiency, heart failure, immunodeficiency, or a combination thereof. Any of the aforementioned pharmaceutical composition comprising the bioactive agents and the LCFA can be suitable. The aforementioned process for producing the pharmaceutical composition can be suitable.

The pharmaceutical composition of this disclosure can comprise one or more C10 to C40 LCFAs that each can have a free carboxylic acid or a salt thereof and can have the advantage of delivering the bioactive agent into cells, tissues or organs that have the expression of FATP4. As disclosed herein, short chain fatty acid (SCFA), such as a fatty acid have 8 or less carbon atoms are not effectively transported or delivered in vivo (FIG. 10).

The present disclosure provides a form of drug conjugates that can be effectively transported across the small intestinal epithelium through a cell membrane transporter, such as FATP4. The small intestine is the major organ for absorption of nutrients including dietary lipids that are composed of mainly long-chain fatty acids (consisting of 10 or more carbons in the molecule) and glycerol. Transport of LCFA across the apical membrane of small intestinal epithelial cells is mediated by the FATP4 transporter. Since FATP4 can also recognize LCFA-drug conjugates, it provides an advantage for effective absorption of drug molecules from the lumen of small intestine.

The present invention also provides a method for treatment of human diseases with orally delivered bioactive or therapeutic agents. In one particular embodiment, the therapeutic peptides retain their activities after conjugation with LCFA. The LCFA-peptide conjugates can be packaged into enteric-coated formulations. The resulting oral peptide drugs are protected from acidic fluid and digestive enzymes in the stomach. Once the particles reach the small intestine, the pH-responsive coating detach from the package, and the drug conjugates are released to the pH-neutral small intestine lumen where they are transported across the small intestinal epithelial cells as mediated by the FATP4 transporter. In another particular embodiment, a small molecule drug is chemically conjugated to LCFA. The LCFA-drug conjugate can be orally delivered either as a free drug or in an enteric-coated formulation. The drug conjugate is absorbed in the small intestine, and the active drug molecule is cleaved from LCFA afterwards. In an additional particular embodiment, a nanoparticle can be conjugated to the side chain of LCFA either directly or through a LCFA-conjugated drug. The LCFA-nanoparticle conjugates can be packaged in an enteric-coated formulation. This generates an orally deliverable form of nanoparticles. LCFA-nanoparticle can serve as a therapeutic agent, a diagnostic agent, or a carrier for other drugs, such as oligonucleotides.

The instant disclosure now will be exemplified in the following non-limiting examples.

### EXAMPLES

The present invention is further defined in the following Examples. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various uses and conditions.

### Example 1

### Synthesis of LCFA-Ex4 Conjugates

Hexadecanedioic acid serves as an example for LCFA in drug conjugation. Site-specific conjugation of Exendin-4 (Ex4) to hexadecanedioic acid was carried out in a two-step process. In the first step, hexadecanedioic acid was mixed with N-hydroxysuccinimide (NHS) in the presence of N-(3-Dimethylaminopropyl)-N′-ethylcarbodiimide hydrochloride (EDC) to form 16-[2,5-Dioxopyrrolidin-1-yl]oxy)-16-oxohexadecanedioic acid. The resulting product was then mixed with Ex4 in presence of N,N-Diisopropylethylamine (DIPEA) in the second step. The reaction is stopped by adding 1% trifluoro acetic acid (TFA) in the reaction mixture.

The following dicarboxylic acids were used in conjugation: octanedioic acid (C₈), dodecanedioic acid (C₁₂), hexadecanedioic acid (C₁₆) and octadecanedioic acid (C₁₈) were used to synthesize LCFA-Ex4. Both Lys¹² and Lys²⁷ residues could be covalently conjugated with the LCFA (FIG. 6A-FIG. 6C). Lys²⁷ was preferred over Lys¹². In a controlled experiment, a large quantity of Ex4-Lys²⁷-LCFA can be generated (FIG. 7 and FIG. 8).

### Example 2

### Purification and Characterization of Ex4-Lys27-LCFA

Reaction mixture of Ex4 and LCFA conjugation was applied for separation in an Agilent semipreparative HPLC instrument (Agilent Infinity 1260) with a ZORBAX 300SB-C18 column. Eluents for separation were 0.1% TFA in Solvent A and acetonitrile in 0.1% TFA in Solvent B. The collected samples were lyophilized to remove acetonitrile. The purified product was characterized with HPLC (FIG. 8) and confirmed with ESI-MS mass spectrometry (FIG. 9).

### Example 3

### Uptake of Fatty Acid-Ex4 Conjugate by Caco-2 Cells

Coco-2 cells were seeded in cell culture plates. Cells were incubated with either fluorescently labeled Ex4-Lys²⁷-SCFA (C8) or one of the fluorescently labeled Ex4-Lys²⁷-LCFA having a C₁₂, C₁₆ and C₁₈ LCFA. After incubation for one hour, cells were washed with PBS to remove fatty acid-conjugated Ex4. A plate reader was applied to measure fluorescent intensity in each well in the cell culture plate, and the results were compared (FIG. 10).

### Example 4

### Evaluation of Glucose-Lowering Capacity from Ex4-Lys²⁷-LCFA in Acid-Neutralized Mice

Intraperitoneal glucose tolerance test (GTT) was performed in male C57BL/6 mice. Briefly, 6 to 12-week-old male C57BL/6 mice were fasted overnight and treated by oral gavage with 100 μL 1M NaHCO3 to neutralize acid in the gastric fluid. One minute later, the mice were treated with PBS, 100 μg Ex4, or 100 μg Ex4-Lys²⁷-LCFA by oral gavage, respectively. All mice were then treated with 2 g/kg glucose by intraperitoneal injection 1 hour later. Blood samples were collected to measure blood glucose levels. Area under the curve (AUC) of blood glucose was calculated to evaluate hypoglycemic response to each treatment (FIG. 11).

### Example 5

### Evaluation of Glucose-Lowering Activity from GLP-1-Lys26-LCFA in Acid-Neutralized Mice

Intraperitoneal GTT was performed in male C57BL/6 mice. Briefly, 6 to 12-week-old male C57BL/6 mice were fasted overnight and treated with 100 μL 1M NaHCO3 to neutralize acid in the gastric fluid. One minute later, the mice were treated with PBS, 100 μg GLP-1 analogue, or 100 μg GLP-1-Lys²⁶-LCFA by oral gavage, respectively. All mice were then treated with 2 g/kg glucose by intraperitoneal injection 1 hour later. Blood samples were collected to measure blood glucose levels. Area under the curve (AUC) of blood glucose was calculated to evaluate hypoglycemic response to each treatment (FIG. 12).

### Example 6

### Glucose-Lowering Activity from GLP-1-Lys²⁶-LCFA in Enteric-Coated Formulations

Intraperitoneal GTT was performed in male C57BL/6 mice. Briefly, 6 to 12-week-old male C57BL/6 mice were fasted overnight and treated with PBS control, GLP-1-Lys²⁶-LCFA packaged in capsules, or free GLP-1-Lys²⁶-LCFA by oral gavage, respectively (FIG. 13). GLP-1-Lys²⁶-LCFA packaged in Eudragit L-100-coated SiO2 formulation was assayed at 3 different dosage level, 10 μg, 30 μg or 100 μg, respectively (FIG. 14). All mice were then treated with 2 g/kg glucose by intraperitoneal injection 1 hour later. Blood samples were collected to measure blood glucose levels. Area under the curve (AUC) of blood glucose was calculated to evaluate hypoglycemic response to each treatment. The results demonstrate that the enteric-coated GLP1-Lys²⁶-LCFA conjugate is effective in controlling blood glucose level.

### Example 7

### Comparison of Ibrutinib Uptake Based on Changes in Plasma Ibrutinib Concentration

LCFA-conjugated ibrutinib was synthesized and characterized (FIG. 15A-FIG. 15C). To compare drug uptake between free ibrutinib and LCFA-ibrutinib, mice were treated with an equal amount of free ibrutinib or LCFA-ibrutinib. Blood samples were collected at different time points, and analyzed for ibrutinib content by HPLC. Area under the curve (AUC) of ibrutinib was calculated to evaluate plasma drug concentration (FIG. 16A-FIG. 16B). Date showed that the blood level of ibrutinib was higher with LCFA-ibrutinib (AUC: 6.42837) than that of free ibrutinib (AUC: 2.01081).

### Example 8

### Synthesis of LCFA-Conjugated MicroRNA Mimic

Click chemistry was applied to conjugate LCFA to microRNA-34a-3p (miR-34a-3p). Only the anti-sense strand of the miR-34a mimic duplex was chemically conjugated to LCFA and was annealed to the sense strand to form a double stranded RNA-LCFA complex (formula 4) (FIG. 17A). The single stranded structure (formula 5) is shown in FIG. 17B. Briefly, 3-miR-oxy-2-[3-(3-propargyloxypropanamido) propanamido] propyl-1-hydroxy was conjugated with 16-azidohexadecanoic acid via 1,4-disubstituted 1,2,3-triazole. The resulting product was purified. It was then mixed with miR-34a-5p, and the mixture was heated to 95° C. and then gradually cooled down to allow proper annealing of the two single strand nucleotide oligos.

### Example 9

### Knockdown of c-Myc Expression by LCFA-Conjugated MicroRNA-34a Mimic

Caco-2 cells were seeded in 6-well cell culture plates, and treated (without any transfection reagent) with 200 nM free scramble siRNA (Scr), 200 nM free miR-34a mimic (miR-34a), or 200 nM LCFA-conjugated miR-34a (FA-miR-34a), respectively. Cells were harvested 3 days later, and lysed to prepare protein samples. Equal amount of protein samples was loaded in 10% SDS-polyacrylamide gels for Western blot analysis. The c-Myc protein was detected with an anti-c-Myc antibody. c-Myc expression levels were detected after both a short-time exposure (short expo) and relatively long-time exposure (long expo). β-Actin was served as an expression control (FIG. 18).

### REFERENCES

1. Byrd, J. C., et al., Targeting BTK with ibrutinib in relapsed chronic lymphocytic leukemia. N Engl J Med, 2013. 369(1): p. 32-42.
2. Burger, J. A., et al., Ibrutinib as Initial Therapy for Patients with Chronic Lymphocytic Leukemia. New England Journal of Medicine, 2015. 373(25): p. 2425-2437.
3. Dimopoulos, M. A., et al., Phase 3 Trial of Ibrutinib plus Rituximab in Waldenstrom's Macroglobulinemia. N Engl J Med, 2018. 378(25): p. 2399-2410.
4. de Vries, R., et al., Stable isotope-labelled intravenous microdose for absolute bioavailability and effect of grapefruit juice on ibrutinib in healthy adults. Br J Clin Pharmacol, 2016. 81(2): p. 235-45.
5. Blanco, E., H. Shen, and M. Ferrari, Principles of nanoparticle design for overcoming biological barriers to drug delivery. Nat Biotechnol, 2015. 33(9): p. 941-51.
6. Granhall, C., et al., Safety and Pharmacokinetics of Single and Multiple Ascending Doses of the Novel Oral Human GLP-1 Analogue, Oral Semaglutide, in Healthy Subjects and Subjects with Type 2 Diabetes. Clin Pharmacokinet, 2019. 58(6): p. 781-791.
7. Davies, M., et al., Effect of Oral Semaglutide Compared With Placebo and Subcutaneous Semaglutide on Glycemic Control in Patients With Type 2 Diabetes: A Randomized Clinical Trial. JAMA, 2017. 318(15): p. 1460-1470.
8. Wilhelm, S., et al., Analysis of nanoparticle delivery to tumours. Nature Reviews Materials, 2016. 1(5).
9. Park, K., Facing the Truth about Nanotechnology in Drug Delivery. Acs Nano, 2013. 7(9): p. 7442-7447.
10. Moroz, E., S. Matoori, and J. C. Leroux, Oral delivery of macromolecular drugs: Where we are after almost 100 years of attempts. Adv Drug Deliv Rev, 2016. 101: p. 108-121.
11. Menzel, C., et al., In vivo evaluation of an oral self-emulsifying drug delivery system (SEDDS) for exenatide. J Control Release, 2018. 277: p. 165-172.
12. Buckley, S. T., et al., Transcellular stomach absorption of a derivatized glucagon-like peptide-1 receptor agonist. Sci Transl Med, 2018. 10(467).
13. Pridgen, E. M., et al., Transepithelial transport of Fc-targeted nanoparticles by the neonatal fc receptor for oral delivery. Sci Transl Med, 2013. 5(213): p. 213ra167.
14. Schaap, F. G., M. Trauner, and P. L. Jansen, Bile acid receptors as targets for drug development. Nat Rev Gastroenterol Hepatol, 2014. 11(1): p. 55-67.
15. Petrus, A. K., T. J. Fairchild, and R. P. Doyle, Traveling the vitamin B12 pathway: oral delivery of protein and peptide drugs. Angew Chem Int Ed Engl, 2009. 48(6): p. 1022-8.
16. Bouchard-Mercier, A., et al., The metabolic signature associated with the Western dietary pattern: a cross-sectional study. Nutrition Journal, 2013. 12.
17. Stahl, A., et al., Identification of the major intestinal fatty acid transport protein. Mol Cell, 1999. 4(3): p. 299-308.
18. Kalivianakis, M., et al., Detection of impaired intestinal absorption of long-chain fatty acids: validation studies of a novel test in a rat model of fat malabsorption. Am J Clin Nutr, 2000. 72(1): p. 174-80.

### Numbered Embodiments of the Disclosure

Other subject matter contemplated by the present disclosure is set out in the following numbered embodiments (the term “composition” used herein refers to a “pharmaceutical composition”):

(1) A composition comprising a compound of the formula 6:

D-L-F

or a salt thereof, wherein

D is an active pharmaceutical ingredient (API);

F a nutrient that is absorbed in the small intestine; and

L is a linker that connects to D and F.

(2) The composition of embodiment 1, wherein the active pharmaceutical ingredient is a therapeutic protein or peptide.
(3) The composition of embodiment 1, wherein the active pharmaceutical ingredient is a nucleic acid-based therapeutic agent.
(4) The composition of embodiment 1, wherein the active pharmaceutical ingredient is a small molecule compound.
(5) The composition of embodiment 1, wherein the nutrient is a long-chain fatty acid (LCFA).
(6) The composition of embodiment 5, wherein the long-chain fatty acid has an aliphatic chain of 11 carbon atoms or longer.

(7) The composition of embodiment 6, wherein the long-chain fatty acid is a linear fatty acid.

(8) The composition of embodiment 6, wherein the long-chain fatty acid is a branched fatty acid.
(9) The composition of embodiment 6, wherein the long-chain fatty acid is a saturated fatty acid.
(10) The composition of embodiment 6, wherein the long-chain fatty acid is an unsaturated fatty acid.
(11) The composition of embodiment 10, wherein the unsaturated fatty acid is a mono-unsaturated fatty acid.
(12) The composition of embodiment 10, wherein the unsaturated fatty acid is a poly-unsaturated fatty acid.
(13) The composition of embodiment 1, wherein the linker is chemically conjugated to the active pharmaceutical ingredient and the long-chain fatty acid.
(14) The composition of embodiment 13, wherein the linker is an ether, an ester, an amide, a carbamate, a disulfide bond, or a triazole.
(15) The composition of embodiment 13, wherein the linker is a combination of an ether, an ester, an amide, a carbamate, a disulfide bond, or a triazole.
(16) The composition of embodiment 15, wherein the ends of the linker is separated with a spacer.
(17) The composition of embodiment 1, wherein the linker is a chemically active group in the long-chain fatty acid.
(18) The composition of embodiment 1, wherein the linker is a chemically active group in the active pharmaceutical ingredient.
(19) The composition of embodiment 1, wherein the linker is formed between the active pharmaceutical ingredient and long-chain fatty acid after chemical conjugation.
(20) The composition of a drug formulation comprising:

at least one active pharmaceutical ingredient (API); and

at least one enteric coating agent,

wherein at least one active pharmaceutical ingredient is conjugated to a long-chain fatty acid.

(21) The composition of embodiment 20, wherein the active drug molecules are packaged in an enteric-release formulation.
(22) The composition of embodiment 21, wherein the drug formulation is an enteric-coated capsule.
(23) The composition of embodiment 21, wherein the drug formulation is an enteric-coated tablet.
(24) The composition of embodiment 21, wherein the drug formulation is an enteric-coated drug particle suspension.
(25) The composition of embodiment 21, wherein the drug formulation is an enteric-coated drug powder.
(26) The compositions of any one of the embodiments above, wherein the bioactive agent can comprise a glucagon or a derivative thereof, a glucagon analogue or a derivative thereof, a glucagon-like peptide 1 (GLP-1) or a derivative thereof, exendin-4 or a derivative thereof, insulin or a derivative thereof, human brain natriuretic peptide or a derivative thereof, octreotide or a derivative thereof, human vasoactive intestinal peptide (VIP) or a derivative thereof, or a combination thereof.
(27) The compositions of any one of the embodiments above, wherein the bioactive agent can comprise palmitate-conjugated microRNA-34a (miR-34a) mimic or a derivative thereof, Let-7 mimic or a derivative thereof, miR-155 mimic or a derivative thereof, human IRE1 siRNA or a derivative thereof, or a combination thereof.
(28) A method of treating human diseases, comprising a step of oral administering in need thereof a composition comprising:

at least one active pharmaceutical ingredient (API); and

at least one enteric coating agent,

wherein the at least one active pharmaceutical ingredient is conjugated to a nutrient that is absorbed in the small intestine.

(29) The method of embodiment 28, wherein the active pharmaceutical ingredient is chemically conjugated to a long-chain fatty acid.
(30) The method of embodiment 29, wherein the active pharmaceutical ingredient is a therapeutic protein or peptide.
(31) The method of embodiment 29, wherein the active pharmaceutical ingredient is a nucleic acid-based therapeutic agent.
(32) The method of embodiment 29, wherein the active pharmaceutical ingredient is a small molecule compound.
(33) The method of embodiment 28, wherein the long-chain fatty acid-conjugated active pharmaceutical ingredient is prepared in an enteric-release formulation for drug absorption in the small intestine.
(34) The method of embodiment 33, wherein the drug formulation is an enteric-coated capsule.
(35) The method of embodiment 33, wherein the drug formulation is an enteric-coated tablet.
(36) The method of embodiment 33, wherein the drug formulation is an enteric-coated drug particle suspension.
(37) The method of embodiment 33, wherein the drug formulation is an enteric-coated drug powder.
(38) A use of a composition comprising a bioactive agent covalently conjugated to at least one long chain fatty acid (LCFA) for manufacturing a medicament for treating a disease, wherein the long chain fatty acid can comprise a carbon chain having at least 10 carbon atoms, a first chain end and a second chain end connected by said carbon chain, wherein the first chain end can be linked to the bioactive agent and the second chain end can comprise a free carboxylic acid group, and wherein the disease can be a metabolic disorder, a cancer, respiratory disease, depressing, diabetics, infectious disease, hypertensive, allergy, psychotic disorder, neurological disorder, arthritic disease, coagulation disease, ulcer, reflux, nausea, vitamins deficiency, heart failure, immunodeficiency, or a combination thereof.
(39) The use of embodiment 38, wherein the bioactive agent comprises a polypeptide, a small molecule drug, a poly-nucleic acid, or a combination thereof, wherein the long chain fatty acid (LCFA) comprises a C10 to C40 branched or linear saturated fatty acid, C10 to C40 branched or linear unsaturated fatty acid, or a combination thereof, and wherein said long chain fatty acid (LCFA) is linked to said bioactive agent via one or more functional groups that comprise C, N, O, P, S, an ether, an ester, an amide, a carbamate, a disulfide bond, a triazole, or a combination thereof, wherein said functional groups are on said bioactive agent, on said linker when present, or a combination thereof, and wherein said linker comprises 0 to 40 atoms.
(40) The use of embodiments 38-39, wherein the bioactive agent comprises a polypeptide and wherein said linker comprises 0 to 10 atoms and said linker is free from glutamic acid, glutamic acid derivative, glutamine, glutamine derivative, or a combination thereof.
(41) The use of embodiments 38-40, wherein said polypeptide comprises amino acid, modified amino acid, amino acid analogue, or a combination thereof, and said long chain fatty acid (LCFA) is linked to said polypeptide at one or more —N—, —NH—, —NH₂, —S—, —SH, —OH, —COO— or a combination thereof, of one or more residues of said amino acid, modified amino acid, amino acid analogue, or a combination thereof, of said polypeptide.
(42) The use of embodiments 38-41, wherein said bioactive agent comprises a glucagon or a derivative thereof, a glucagon analogue or a derivative thereof, a glucagon-like peptide 1 (GLP-1) or a derivative thereof, exendin-4 or a derivative thereof, insulin or a derivative thereof, human brain natriuretic peptide or a derivative thereof, octreotide or a derivative thereof, human vasoactive intestinal peptide (VIP) or a derivative thereof, or a combination thereof.
(43) The use of embodiments 38-39, wherein the bioactive agent comprises a metabolic drug, a cancer drug, a chemotherapeutic drug, analgesics/antipyretics, anesthetics, antiasthmatics, antidepressants, antidiabetics, antifungal agents antihypertensive agents, antipsychotic agents, antimanic agents, antiarrhythmics, antiarthritic agents, antigout agents, anticoagulants, thrombolytic agents, antifibrinolytic agents, hemorheologic agents, antiplatelet agents, anticonvulsants, antiparkinson agents, antihistamines/antipruritics, agents useful for calcium regulation, antibacterial agents, antiviral agents, antimicrobials, antibiotics, anti-infectives, corticosteroids, thyroid hormones, hypoglycemic agents selected from insulin, recombinant insulin, glyburide, chlorpropamide, glipizide, tolbutamide, tolazamide, or a combination thereof, hypolipidemic agents selected from clofibrate, dextrothyroxine sodium, probucol, lovastatin, niacin or a combination thereof, antiulcer or antireflux agents, antinauseants or antiemetics, vitamins, mitotane, visadine, halonitrosoureas, anthrocyclines, ellipticine, or a combination thereof.
(44) The use of embodiments 38-39, wherein the bioactive agent comprises the poly-nucleic acid comprising a DNA, an RNA, a poly-oligonucleotide, a poly-oligodeoxynucleotide, a derivative thereof, or a combination thereof.
(45) The use of embodiment 44, wherein the bioactive agent comprises palmitate-conjugated microRNA-34a (miR-34a) mimic or a derivative thereof, Let-7 mimic or a derivative thereof, miR-155 mimic or a derivative thereof, human IRE1 siRNA or a derivative thereof, or a combination thereof.

1. A pharmaceutical composition comprising a bioactive agent and at least one long chain fatty acid (LCFA), wherein said long chain fatty acid comprises a carbon chain having at least 10 carbon atoms comprising a first chain end and a second chain end, wherein said first chain end is covalently linked to said bioactive agent directly, or optionally, via a linker, and said second chain end comprises a free carboxylic acid group or a salt thereof.

2. The pharmaceutical composition of claim 1, wherein said bioactive agent comprises a polypeptide, a small molecule drug, a poly-nucleic acid, or a combination thereof, wherein said long chain fatty acid (LCFA) comprises a C10 to C40 branched or linear saturated fatty acid, C10 to C40 branched or linear unsaturated fatty acid, or a combination thereof, and wherein said long chain fatty acid (LCFA) is linked to said bioactive agent via one or more functional groups that comprise C, N, O, P, S, an ether, an ester, an amide, a carbamate, a disulfide bond, a triazole, or a combination thereof, wherein said functional groups are on said bioactive agent, on said linker when present, or a combination thereof, and wherein said linker comprises 0 to 40 atoms.

3. The pharmaceutical composition of claim 2, wherein said bioactive agent comprises a polypeptide and wherein said linker comprises 0 to 10 atoms and said linker is free from glutamic acid, glutamic acid derivative, glutamine, glutamine derivative, or a combination thereof.

4. The pharmaceutical composition of claim 3, wherein said polypeptide comprises amino acid, modified amino acid, amino acid analogue, or a combination thereof, and said long chain fatty acid (LCFA) is linked to said polypeptide at one or more —N—, —NH—, —NH₂, —S—, —SH, —OH, —COO— or a combination thereof, of one or more residues of said amino acid, modified amino acid, amino acid analogue, or a combination thereof, of said polypeptide.

5. The pharmaceutical composition of claim 3, wherein said bioactive agent comprises a glucagon or a derivative thereof, a glucagon analogue or a derivative thereof, a glucagon-like peptide 1 (GLP-1) or a derivative thereof, exendin-4 or a derivative thereof, insulin or a derivative thereof, human brain natriuretic peptide or a derivative thereof, octreotide or a derivative thereof, human vasoactive intestinal peptide (VIP) or a derivative thereof, or a combination thereof.

6. The pharmaceutical composition of claim 5, wherein said pharmaceutical composition comprises said bioactive agent and said long chain fatty acid (LCFA) having the 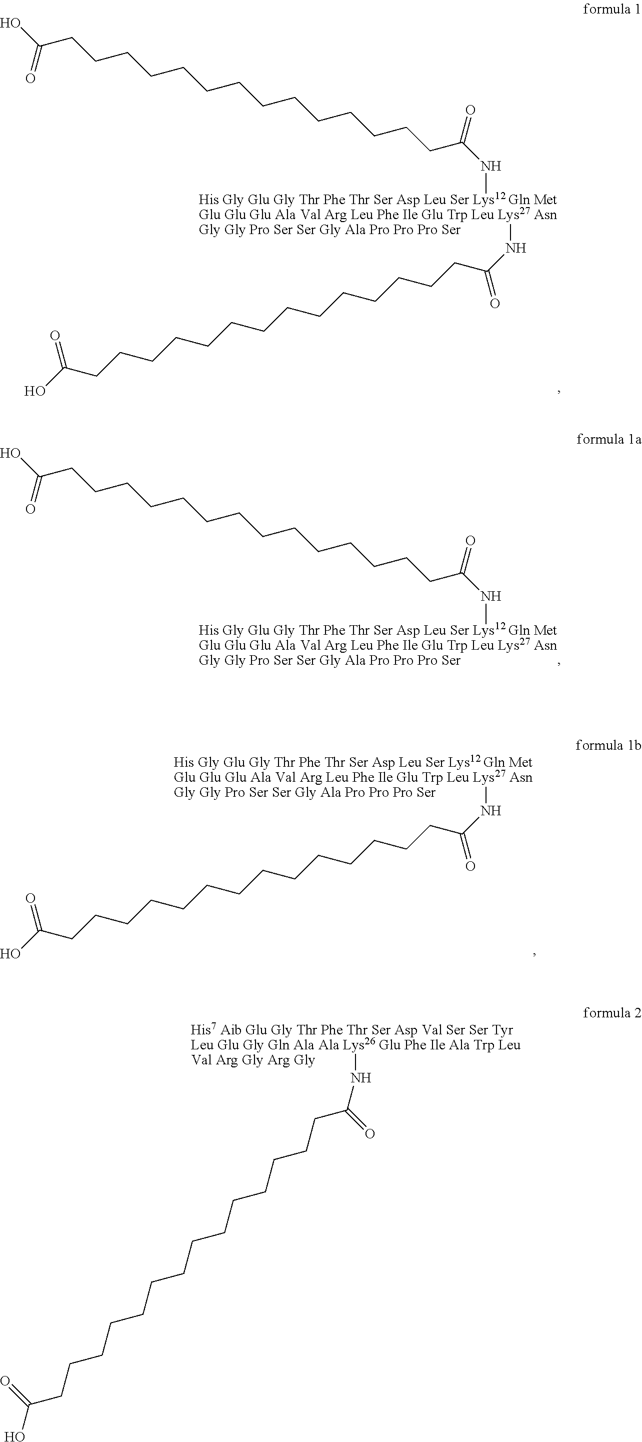
or a combination thereof.

7. The pharmaceutical composition of claim 2, wherein said bioactive agent comprises a metabolic drug, a cancer drug, a chemotherapeutic drug, analgesics/antipyretics, anesthetics, antiasthmatics, antidepressants, antidiabetics, antifungal agents antihypertensive agents, antipsychotic agents, antimanic agents, antiarrhythmics, antiarthritic agents, antigout agents, anticoagulants, thrombolytic agents, antifibrinolytic agents, hemorheologic agents, antiplatelet agents, anticonvulsants, antiparkinson agents, antihistamines/antipruritics, agents useful for calcium regulation, antibacterial agents, antiviral agents, antimicrobials, antibiotics, anti-infectives, corticosteroids, thyroid hormones, hypoglycemic agents selected from insulin, recombinant insulin, glyburide, chlorpropamide, glipizide, tolbutamide, tolazamide, or a combination thereof, hypolipidemic agents selected from clofibrate, dextrothyroxine sodium, probucol, lovastatin, niacin or a combination thereof, antiulcer or antireflux agents, antinauseants or antiemetics, vitamins, mitotane, visadine, halonitrosoureas, anthrocyclines, ellipticine, or a combination thereof.

8. The pharmaceutical composition of claim 7, wherein said pharmaceutical composition comprises said bioactive agent and said long chain fatty acid (LCFA) having the

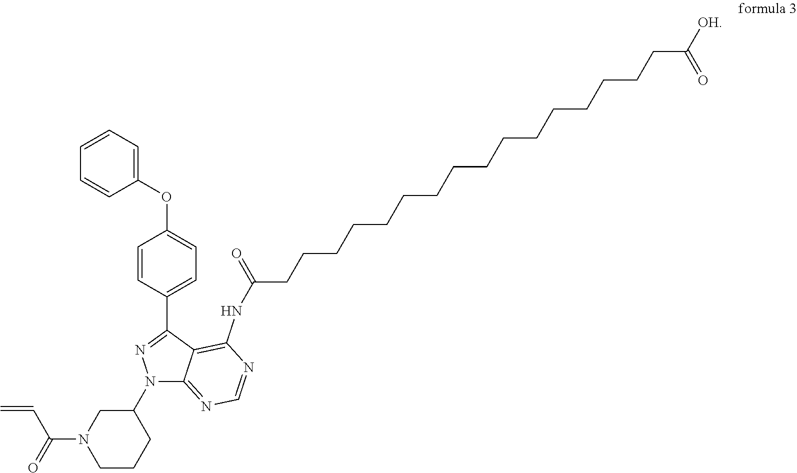 [SMILES: C=CC(=O)N1CCCC(n2nc(-c3ccc(Oc4ccccc4)cc3)c3c(NC(=O)CCCCCCCCCCCCCCCCC(=O)O)ncnc32)C1]

9. The pharmaceutical composition of claim 2, wherein said bioactive agent comprises said poly-nucleic acid comprising a DNA, an RNA, a poly-oligonucleotide, a poly-oligodeoxynucleotide, a derivative thereof, or a combination thereof.

10. The pharmaceutical composition of claim 9, wherein said long chain fatty acid (LCFA) is linked to said poly-nucleic acid at a 3′-end nuclei acid residual of said poly-nucleic acid.

11. The pharmaceutical composition of claim 9, wherein each of said DNA, RNA, poly-oligonucleotide, poly-oligodeoxynucleotide, derivative thereof, or a combination thereof, is a single strand poly-nucleic acid, and wherein said pharmaceutical composition further comprises a complementary strand of said poly-nucleic acid.

12. The pharmaceutical composition of claim 11, wherein said pharmaceutical composition comprises said bioactive agent and said long chain fatty acid (LCFA) having the 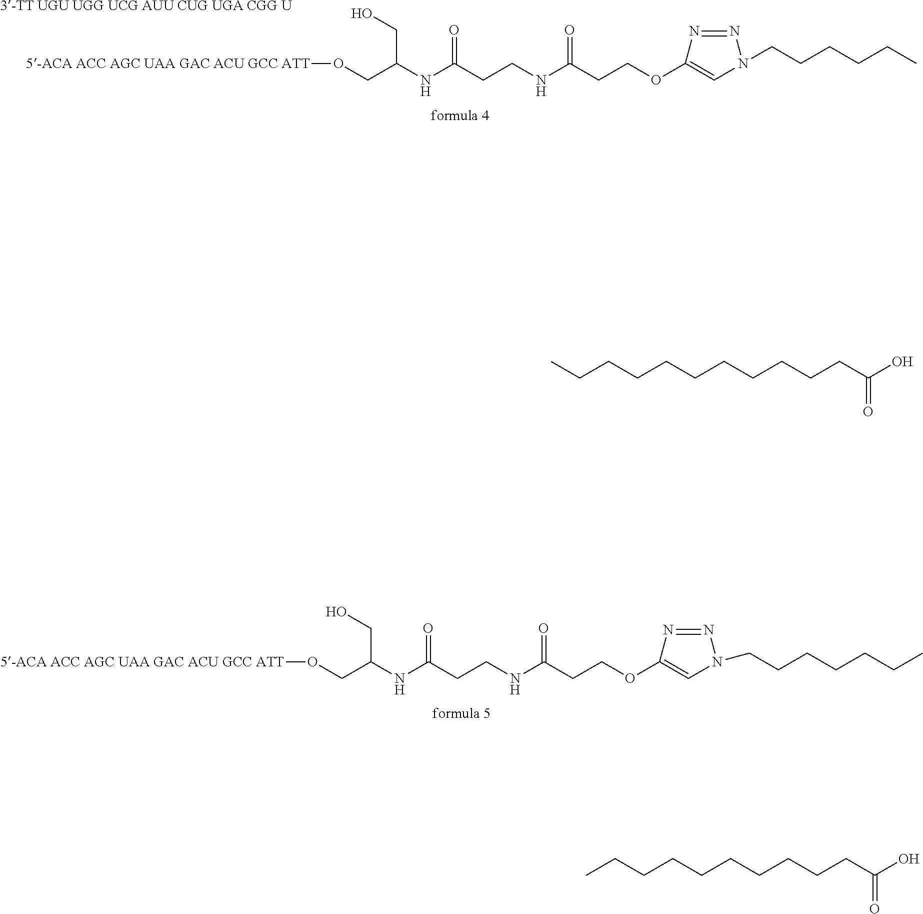
or a combination thereof.

13. The pharmaceutical composition of claim 1 further comprising a pharmaceutically acceptable excipient selected from a detergent, a buffer, a phosphate, a salt, a water, a solvent, a filler, an inorganic compound, an organic compound, a synthetic polymer, a biopolymer, a sugar, a starch, a polysaccharide, SiO₂, Eudragit L-100-coated SiO₂, or a combination thereof.

14. The pharmaceutical composition of claim 1, wherein said pharmaceutical composition is formulated as an enteric-coated capsule, an enteric-coated tablet, an enteric-coated drug particle suspension, an enteric-coated drug powder, or a combination thereof.

15. A process for producing a pharmaceutical composition comprising a bioactive agent covalently conjugated to at least one long chain fatty acid (LCFA), said process comprising:
a) providing a long chain dicarboxylic acid comprising a carbon chain having at least 10 carbon atoms, a first chain end having a first acid group and a second chain end having a second acid group, said first acid group and said second acid group are connected by said carbon chain;
b) reacting said first acid group, optionally via a linker, with said bioactive agent to produce said pharmaceutical composition;
wherein said second chain end in said pharmaceutical composition comprises a free carboxylic acid group or a salt thereof.

16. The process of claim 15, wherein said bioactive agent is selected from a polypeptide, a small molecule drug, a poly-nucleic acid, or a combination thereof, wherein said long chain dicarboxylic acid is selected from C10 to C40 branched or linear saturated dicarboxylic acid, C10 to C40 branched or linear unsaturated dicarboxylic acid, or a combination thereof, and wherein said long chain dicarboxylic acid is linked to said bioactive agent via one or more functional groups that comprise C, N, O, P, S, an ether, an ester, an amide, a carbamate, a disulfide bond, a triazole, or a combination thereof, wherein said functional groups are on said bioactive agent, on said linker when present, or a combination thereof, and wherein said linker comprises 0 to 40 atoms.

17. The process of claim 16, wherein said bioactive agent comprises a polypeptide and wherein said linker comprises 0 to 10 atoms and said linker is free from glutamic acid, glutamic acid derivative, glutamine, glutamine derivative, or a combination thereof.

18. The process of claim 17, wherein said polypeptide comprises amino acid, modified amino acid, amino acid analogue, or a combination thereof, and said long chain fatty acid (LCFA) is linked to said polypeptide at one or more —N—, —NH—, —NH₂, —S—, —SH, —OH, —COO— or a combination thereof, of one or more residues of said amino acid, modified amino acid, amino acid analogue, or a combination thereof, of said polypeptide.

19. The process of claim 17, wherein said bioactive agent comprises a glucagon or a derivative thereof, a glucagon analogue or a derivative thereof, a glucagon-like peptide 1 (GLP-1) or a derivative thereof, exendin-4 or a derivative thereof, insulin or a derivative thereof, human brain natriuretic peptide or a derivative thereof, octreotide or a derivative thereof, human vasoactive intestinal peptide (VIP) or a derivative thereof, or a combination thereof.

20. The process of claim 19, wherein said pharmaceutical composition comprises said bioactive agent and said long chain fatty acid (LCFA) having the 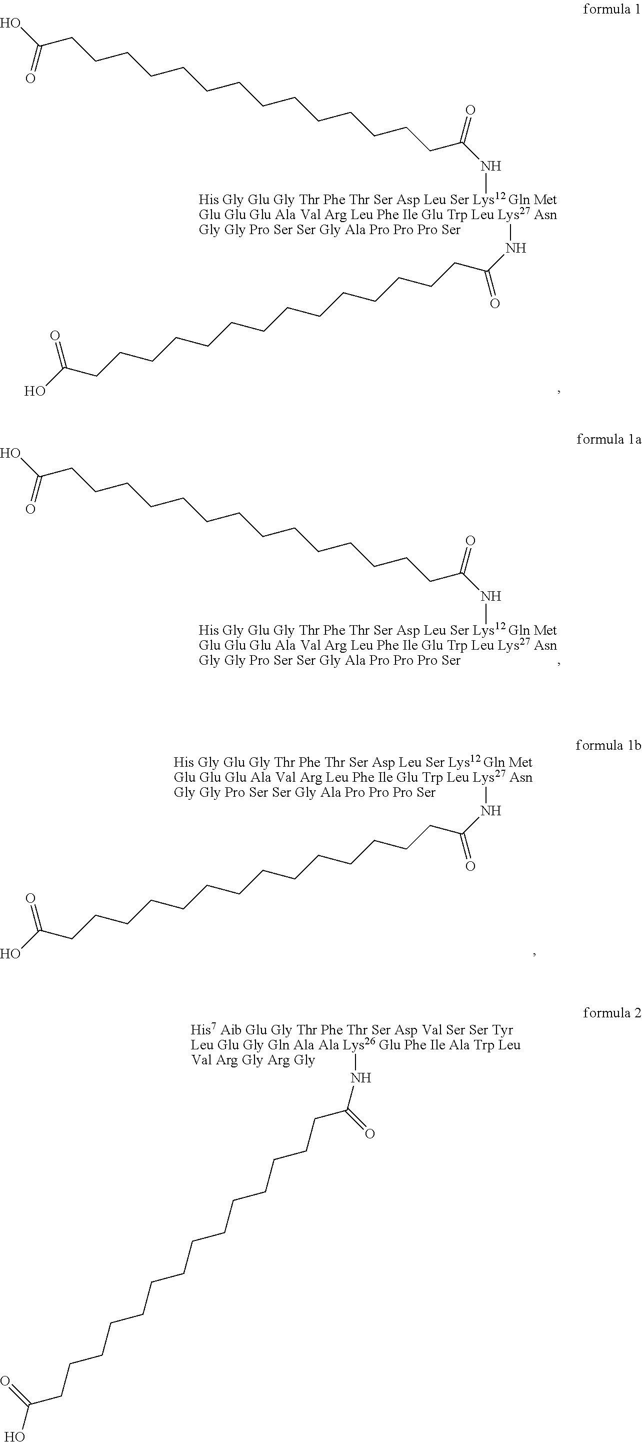
or a combination thereof.

21. The process of claim 16, wherein said bioactive agent comprises a metabolic drug, a cancer drug, a chemotherapeutic drug, analgesics/antipyretics, anesthetics, antiasthmatics, antidepressants, antidiabetics, antifungal agents antihypertensive agents, antipsychotic agents, antimanic agents, antiarrhythmics, antiarthritic agents, antigout agents, anticoagulants, thrombolytic agents, antifibrinolytic agents, hemorheologic agents, antiplatelet agents, anticonvulsants, antiparkinson agents, antihistamines/antipruritics, agents useful for calcium regulation, antibacterial agents, antiviral agents, antimicrobials, antibiotics, anti-infectives, corticosteroids, thyroid hormones, hypoglycemic agents selected from insulin, recombinant insulin, glyburide, chlorpropamide, glipizide, tolbutamide, tolazamide, or a combination thereof, hypolipidemic agents selected from clofibrate, dextrothyroxine sodium, probucol, lovastatin, niacin or a combination thereof, antiulcer or antireflux agents, antinauseants or antiemetics, vitamins, mitotane, visadine, halonitrosoureas, anthrocyclines, ellipticine, or a combination thereof.

22. The process of claim 21, wherein said pharmaceutical composition comprises said bioactive agent and said long chain fatty acid (LCFA) having the

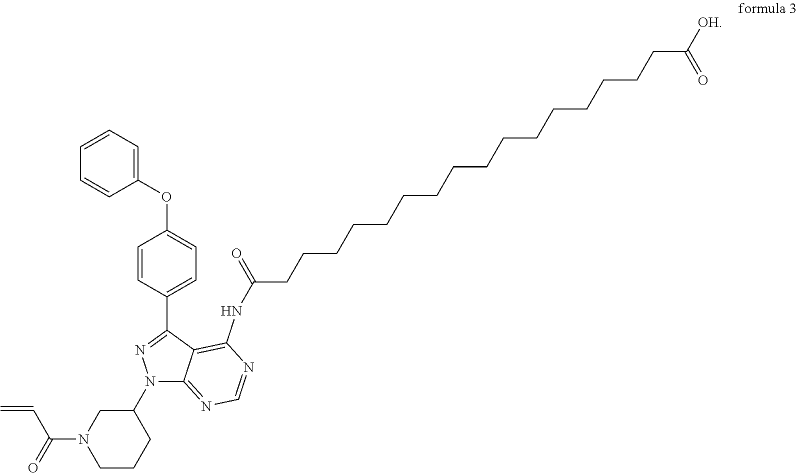 [SMILES: C=CC(=O)N1CCCC(n2nc(-c3ccc(Oc4ccccc4)cc3)c3c(NC(=O)CCCCCCCCCCCCCCCCC(=O)O)ncnc32)C1]

23. The process of claim 16, wherein said bioactive agent comprises said poly-nucleic acid comprising a DNA, an RNA, a poly-oligonucleotide, a poly-oligodeoxynucleotide, a derivative thereof, or a combination thereof.

24. The process of claim 23, wherein said long chain fatty acid (LCFA) is linked to said poly-nucleic acid at a 3′-end nuclei acid residual of said poly-nucleic acid.

25. The process of claim 23, wherein each of said DNA, RNA, poly-oligonucleotide, poly-oligodeoxynucleotide, derivative thereof, or a combination thereof, is a single strand poly-nucleic acid, and wherein said pharmaceutical composition further comprises a complementary strand of said poly-nucleic acid.

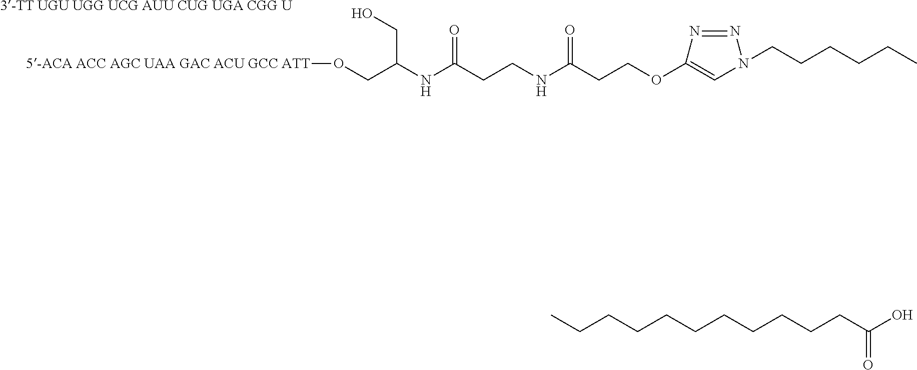

26. The process of claim 25, wherein said pharmaceutical composition comprises said bioactive agent and said long chain fatty acid (LCFA) having the 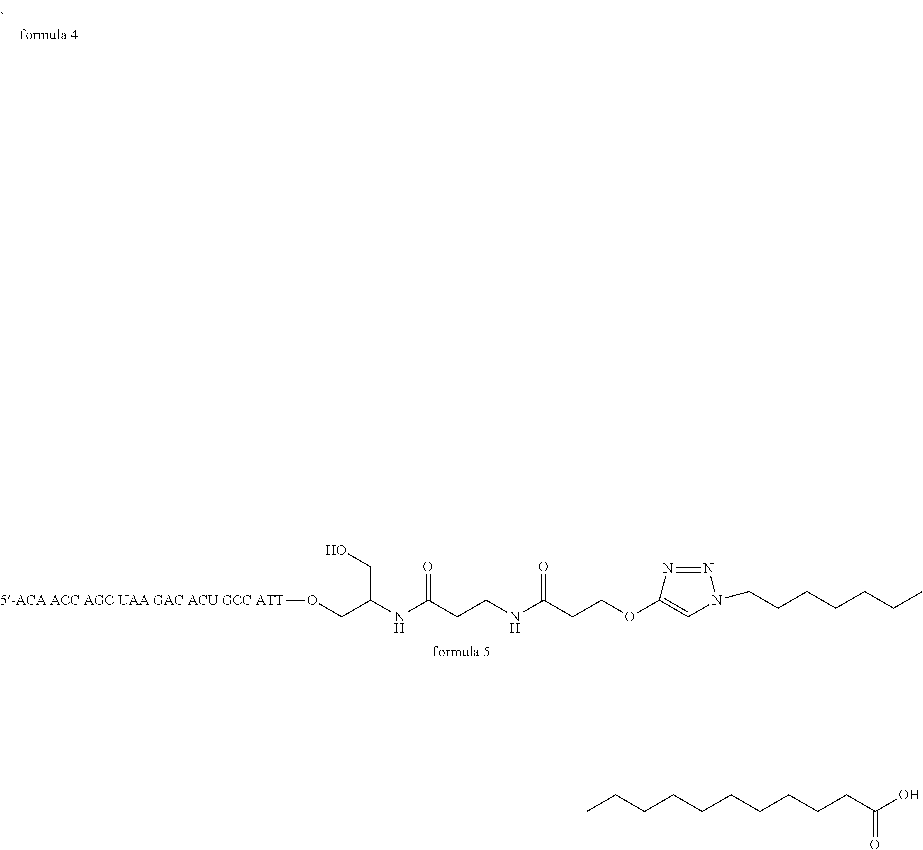
or a combination thereof.

27. The process of claim 15 further comprising mixing a pharmaceutically acceptable excipient selected from a detergent, a buffer, a phosphate, a salt, a water, a solvent, a filler, an inorganic compound, an organic compound, a synthetic polymer, a biopolymer, a sugar, a starch, a polysaccharide, SiO₂, Eudragit L-100-coated SiO₂, or a combination thereof, with said bioactive agent and said long chain fatty acid (LCFA).

28-29. (canceled)
